(19) United States
(12) Patent Application Publication
(10) Pub. No.: US 2026/0272901 A1
(43) Pub. Date: Sep. 17, 2026

(54) SELECTIVE ALPHA-2C ADRENERGIC RECEPTOR AGONISTS AND USES THEREOF

(71) Applicant: AlpheraBio LLC, Andover, MA (US)

(72) Inventors: Lingyu ZHU, Pawcatuck, CT (US); Zhiqiang CHENG, Ellicott City, MD (US)

(21) Appl. No.: 19/564,001

(22) Filed: Mar. 11, 2026

Related U.S. Application Data
(60) Provisional application No. 63/770,814, filed on Mar. 12, 2025.
(60) Provisional application No. 63/812,663, filed on May 27, 2025.

(51) Int. Cl. A61K 31/4409; A61K 31/4436; A61K 31/4439; A61K 31/444; A61K 31/4725; A61K 31/506; A61P 37/06; C07D 213/74; C07D 401/12; C07D 405/12; C07D 409/12; C07D 413/12; C07D 413/14

(52) U.S. Cl. CPC A61K 31/4409; A61K 31/4436; A61K 31/4439; A61K 31/444; A61K 31/4725; A61K 31/506; A61P 37/06; C07D 213/74; C07D 401/12; C07D 405/12; C07D 409/12; C07D 413/12; C07D 413/14

(57) ABSTRACT

The present disclosure relates to selective alpha-2C adrenergic receptor agonists and uses thereof. In particular, the present disclosure relates to compounds of formula (I). These compounds can be useful as alpha-2C adrenergic receptor agonists for the treatment or prevention of disease thereof. In addition, the present disclosure describes the first discovered therapeutic use of these compounds against Tumor Necrosis Factor-α (TNF-α)-mediated inflammatory and autoimmune diseases, since these compounds can significantly inhibit lipopolysaccharide (LPS)-induced inflammatory responses and effectively down-regulate the expression, production and secretion of TNF-α.

### CROSS-REFERENCE TO RELATED APPLICATION

This application claims priority to U.S. Patent Application No. 63/770,814, filed on Mar. 12, 2025, and U.S. Patent Application No. 63/812,663, filed on May 27, 2025, the disclosures of which are incorporated herein by reference in their entireties.

### FIELD OF THE DISCLOSURE

The present disclosure belongs to the field of medicinal chemistry and pharmaceutical technology. The present disclosure describes novel selective alpha-2C adrenergic receptor (ADRA2C) agonists and uses thereof. In particular, the present disclosure relates to compounds of formula (I), which can be useful as alpha-2C adrenergic receptor agonists for the treatment or prevention of disease thereof. In addition, it describes their first medical use in the preparation of a medicament for the treatment of Tumor Necrosis Factor-α (TNF-α)-mediated inflammatory and autoimmune diseases in humans and animals.

### BACKGROUND OF THE DISCLOSURE

Alpha-2 adrenergic receptor (α2-AR) agonists have been widely utilized in clinical settings for their analgesic, anti-inflammatory, and vasoconstrictive properties. These agents play crucial roles in pain management, sedation, and cardiovascular regulation. However, most α2-AR agonists, such as clonidine and dexmedetomidine, activate the α2A subtype, leading to significant central effects, including sedation, bradycardia, hypotension, and hypothermia. These adverse effects have severely limited the broader therapeutic potential of α2 agonists.

Selective activation of the α2C subtype (ADRA2C) without affecting α2A subtype could provide therapeutic benefits of α2-AR agonists while avoiding these dose-limiting side effects. α2C receptors are primarily involved in neurotransmitter release modulation, neuroprotection, and vasoconstriction, making selective α2C adrenergic receptor agonists promising candidates for multiple conditions. The inventors of the present disclosure have developed selective ADRA2C agonists, which lacks α2A activity, represent a potential breakthrough in expanding the clinical utility of α2 agonists while minimizing cardiovascular and sedative risks.

In addition, the inventors of the present disclosure have discovered that these novel compounds can be useful against TNF-α-mediated inflammatory and autoimmune diseases, because they can significantly inhibit lipopolysaccharide (LPS)-induced inflammatory responses and effectively down-regulate the expression, production and secretion of TNF-α.

TNF-α is a core pro-inflammatory cytokine that plays a key role in the occurrence and development of various inflammatory and autoimmune diseases. TNF-α is a well-recognized and validated therapeutic target for rheumatoid arthritis, inflammatory bowel disease, psoriasis, ankylosing spondylitis, sepsis, acute lung injury and other related disorders.

At present, the mainstream anti-TNF-α drugs on the market are biological products, including adalimumab (Humira), infliximab, etanercept, golimumab and certolizumab pegol. These biological drugs have achieved remarkable clinical efficacy, but they still have obvious limitations: high production cost, need for injection administration, poor oral bioavailability, low tissue penetration, potential immunogenicity and side effects. In particular, their application in veterinary medicine is extremely limited due to cost and stability.

Some small-molecule drugs have also been reported to reduce TNF-α production or signaling through modulation of intracellular pathways or upstream regulatory mechanisms. For example, agents such as thalidomide, apremilast, and JAK inhibitors have been shown to decrease TNF-α levels indirectly as part of their broader immunomodulatory effects. In addition, several experimental small-molecule compounds, including apilimod, SPD-304 and TFP-UA, have been investigated for their ability to modulate TNF-α activity in preclinical studies. However, most direct small-molecule modulators of TNF-α remain at the research or early clinical stage.

Accordingly, there is an urgent clinical and market need for novel, safe, stable, and efficient small-molecule TNF-α inhibitors suitable for both human and veterinary applications. The present invention addresses this unmet need.

### BRIEF SUMMARY OF THE DISCLOSURE

In one general aspect, the present disclosure relates to a compound of formula (I):

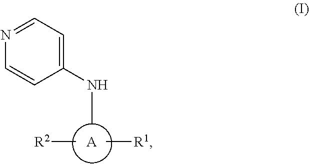

or a stereoisomer, tautomer, pharmaceutically acceptable salt or a solvate thereof,
wherein,
ring A is a bicyclic ring;
R¹ is chosen from —C₀₋₁₂ haloalkyl, —C(O)—C₀₋₁₂ alkyl, —C(O)—C₀₋₁₂ haloalkyl, —C(O)-aryl, —C(O)-heteroaryl, —C₀₋₁₂ alkylene-COOH, —C(O)—NR³R^{3′}, —CN, —NR³R^{3′}, —NC(O)R³R^{3′}, —NR³—SO₂—R^{3′}, —O—C₀₋₁₂ alkyl, —SO₂—R³, —SO₂—NR³R^{3′}, —C₀₋₁₂ alkylene-C₂₋₁₂ heteroaryl, and —C₀₋₁₂ alkylene-C₂₋₁₂ heterocyclyl;
R² is chosen from hydrogen, halogen, haloalkyl, and alkyl; and
each of R³ and R^{3′} is independently hydrogen, haloalkyl, or alkyl.

In some embodiments, the compound of formula (I) is a compound of formula (I-1):

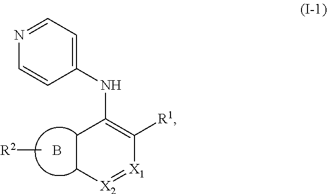

wherein,
   X₁ is N or CH;
   X₂ is N or CH; and
   ring B is aryl or heteroaryl.

In certain embodiments, both X₁ and X₂ are CH, or both X₁ and X₂ are N.

In certain embodiments, X₁ is CH and X₂ is N, or X₁ is N and X₂ is CH.

In certain embodiments, ring B is an aryl, such as phenyl.

In certain embodiments, ring B is a heteroaryl, particularly C₂₋₁₂ heteroaryl, such as pyridine, furan, or thiophene.

In some embodiments, R² is hydrogen. In other embodiments, R² is halogen, such as fluoride, chloride, or bromide.

In some embodiments, wherein the ring A is

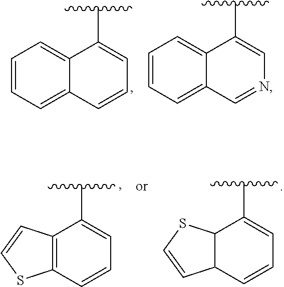

In another aspect, the present disclosure relates to a pharmaceutical composition comprising a compound as described herein or a stereoisomer, tautomer, pharmaceutically acceptable salt or a solvate thereof, and a pharmaceutically acceptable carrier.

In some embodiments, the pharmaceutical composition can be prepared into suitable dosage forms for clinical and veterinary use.

In another aspect, the present disclosure relates to the use of a selective alpha-2C adrenergic receptor (ADRA2C) agonist such as a compound as described herein or a stereoisomer, tautomer, pharmaceutically acceptable salt or a solvate thereof, for treating or preventing a disease, including nasal congestion, chronic rhinitis, neuropathic pain, inflammatory pain, Raynaud's phenomenon, hypotensive shock, cognitive impairment and neurodegenerative diseases (e.g., Alzheimer's disease, Parkinson's disease, Huntington's disease, amyotrophic lateral sclerosis (ALS), multiple sclerosis (MS), ADHD), schizophrenia, alcohol and drug withdrawal syndrome, rosacea, overactive bladder (OAB), glaucoma, and potential tumor growth modulation, etc. in a subject in need thereof.

In another aspect, the present disclosure relates to the use of a selective alpha-2C adrenergic receptor (ADRA2C) agonist such as a compound as described herein or a stereoisomer, tautomer, pharmaceutically acceptable salt or a solvate thereof, for treating or preventing one of inflammatory and autoimmune diseases.

In some embodiments, the selective ADRA2C agonist has an ESR (alpha-2A/alpha-2C) higher than 5, 10, 20, 40, 100, 200, 400, or 800.

In some embodiments, the selective ADRA2C agonist is an alpha-2A adrenergic receptor (ADRA2A) antagonist.

Other features and advantages of the present disclosure are apparent from additional descriptions provided herein, including different examples. The provided examples illustrate different components and methodology useful in practicing the present disclosure. Such examples do not limit the claimed disclosure. Based on the present disclosure, the skilled artisan can identify and employ other components and methodology useful for practicing the present disclosure.

### BRIEF DESCRIPTION OF THE DRAWINGS

The foregoing and other objects, aspects, features, and advantages of exemplary embodiments will become more apparent and may be better understood by referring to the following description taken in conjunction with the accompanying drawings.

FIG. 1 depicts administration of the test compounds significantly reduced plasma TNF-α levels relative to the vehicle control group. In particular, Compounds 7, 12, 13, and 14 each demonstrated inhibitory effects on LPS-induced cytokine production.

### DETAILED DESCRIPTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the disclosure. Such discussion is not an admission that any or all of these matters form part of the prior art with respect to the disclosure.

Unless defined otherwise, all technical and scientific terms used herein have the same meaning as commonly understood to one of ordinary skill in the art to which the present disclosure pertains. Otherwise, certain terms used herein have the meanings as set forth in the specification.

As used herein and in the appended claims, the singular forms “a,” “an,” and “the” include plural reference (one or more) unless the context clearly dictates otherwise.

Unless otherwise indicated, the term “at least” preceding a series of elements is to be understood to refer to every element in the series. For example, the phrase “at least A, B, and C” means that each of A, B, and C is present. The term “at least one of” preceding a series of elements is to be understood to refer to a single element in the series or any combination of two or more elements in the series. For example, the phrase “at least one of A, B, and C” means that only A is present, only B is present, only C is present, both A and B are present, both A and C are present, both B and C are present, or each of A, B, and C is present. Depending on the context, “at least one of” preceding a series of elements can also encompass situations in which any one or more of the elements is present in greater than one instance, e.g., “at least one of A, B, and C” can also encompass situations in which A is present in duplicate alone or further in combination with any one or more of elements B and C.

As used herein, the conjunctive term “and/or” between multiple recited elements is understood as encompassing both individual and conjunctive options. For instance, where two elements are conjoined by “and/or,” a first option refers to the applicability of the first element without the second. A second option refers to the applicability of the second element without the first. A third option refers to, conjunctively, the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

Unless otherwise stated, any numerical value, such as a concentration or a concentration range described herein, are to be understood as being modified in all instances by the term “about.” Thus, a numerical value typically includes ±10% of the recited value. For example, the recitation of “10-fold” includes 9-fold and 11-fold. As used herein, the use of a numerical range expressly includes all possible permutations and combinations of subranges, all individual numerical values within that range, including integers within such ranges and fractions of the values unless the context clearly indicates otherwise.

As used herein, “subject” means any animal, such as a mammal, particularly a human, to whom will be or has been treated by a method described herein. The term “mammal” as used herein, encompasses any mammal. Examples of mammals include, but are not limited to, cows, horses, sheep, pigs, cats, dogs, mice, rats, rabbits, guinea pigs, and non-human primates (NHPs), such as monkeys or apes, humans, etc.

The phrase “pharmaceutically acceptable salt(s)” means those salts of a compound of interest that are safe and effective for topical use in mammals and that possess the desired biological activity. Pharmaceutically acceptable salts include salts of acidic or basic groups present in the specified compounds. Pharmaceutically acceptable acid addition salts include, but are not limited to, hydrochloride, hydrobromide, hydroiodide, nitrate, sulfate, bisulfate, phosphate, acid phosphate, isonicotinate, carbonate, bicarbonate, acetate, lactate, salicylate, citrate, tartrate, propionate, butyrate, pyruvate, oxalate, malonate, pantothenate, bitartrate, ascorbate, succinate, maleate, gentisinate, fumarate, gluconate, glucaronate, saccharate, formate, benzoate, glutamate, methanesulfonate, ethanesulfonate, benzensulfonate, p-toluenesulfonate and pamoate (i.e., 1,1′-methylene-bis-(2-hydroxy-3-naphthoate)) salts. Certain compounds used in the present disclosure can form pharmaceutically acceptable salts with various amino acids. Suitable base salts include, but are not limited to, aluminum, calcium, lithium, magnesium, potassium, sodium, zinc, bismuth, and diethanolamine salts. For a review on pharmaceutically acceptable salts see Berge et al., 66 J. Pharm. Sci. 1-19 (1977), incorporated herein by reference.

As used herein, the term “alkyl” means a saturated, monovalent, unbranched or branched hydrocarbon chain. An alkyl group can be unsubstituted or substituted with one or more suitable substituents. Examples of alkyl groups include, but are not limited to, methyl (Me), ethyl (Et), propyl (e.g., n-propyl, isopropyl), butyl (e.g., n-butyl, isobutyl, tert-butyl), and pentyl (e.g., n-pentyl, isopentyl, neopentyl), etc. An alkyl group can have a specified number of carbon atoms. When numbers appear in a subscript after the symbol “C”, the subscript defines with more specificity the number of carbon atoms which that particular alkyl can contain. For example, “C₁ to C₁₀ alkyl” or “C₁₋₁₀ alkyl” is intended to include alkyl groups having 1, 2, 3, 4, 5, 6, 7, 8, 9, and 10 carbon atoms. Additionally, for example, “C₁ to C₈ alkyl” or “C₁₋₈ alkyl” denotes an alkyl having 1, 2, 3, 4, 5, 6, 7, or 8 carbon atoms.

As used herein, the term “alkenyl” refers to an unbranched or branched hydrocarbon chain containing at least one carbon-carbon double bond. An alkenyl group can be unsubstituted or substituted with one or more suitable substituents. Examples of alkenyl groups include ethenyl, propenyl, butadienyl (including 1,2-butadienyl and 1,3-butadienyl). When numbers appear in a subscript after the symbol “C”, the subscript defines with more specificity the number of carbon atoms which that particular alkenyl can contain. For example, “C₂ to C₁₀ alkenyl” or “C₂₋₁₀ alkenyl” is intended to include alkenyl groups having 2, 3, 4, 5, 6, 7, 8, 9, and 10 carbon atoms. Additionally, for example, “C₂ to C₈ alkenyl” or “C₂₋₈ alkenyl” denotes an alkenyl having 2, 3, 4, 5, 6, 7, or 8 carbon atoms.

As used herein, the term “alkynyl” refers to an unbranched or branched hydrocarbon chain containing at least one carbon-carbon triple bond. An alkynyl group can be unsubstituted or substituted with one or more suitable substituents. The term “alkynyl” also includes those groups having one triple bond and one double bond. When numbers appear in a subscript after the symbol “C”, the subscript defines with more specificity the number of carbon atoms which that particular alkynyl can contain. For example, “C₂ to C₁₀ alkynyl” or “C₂₋₁₀ alkynyl” is intended to include alkynyl groups having 2, 3, 4, 5, 6, 7, 8, 9, and 10 carbon atoms. Additionally, for example, “C₂ to C₈ alkynyl” or “C₂₋₈ alkynyl” denotes an alkynyl having 2, 3, 4, 5, 6, 7, or 8 carbon atoms.

As used herein, the term “cycloalkyl” refers to any stable monocyclic or polycyclic saturated hydrocarbon ring system. A cycloalkyl group can be unsubstituted or substituted with one or more suitable substituents. A cycloalkyl group can have a specified number of carbon atoms. For example, “C₃ to C₆ cycloalkyl” or “C₃₋₆ cycloalkyl” includes cycloalkyl groups having 3, 4, 5, or 6 ring carbon atoms, i.e., cyclopropyl, cyclobutyl, cyclopentyl, and cyclohexyl. Polycyclic cycloalkyls include bridged, fused, and spiro ring structures in which all ring atoms are carbon atoms. A “spiro ring” is a polycyclic ring system in which two rings share one carbon atom, referred to as the “spiro atom,” which is typically a quaternary carbon atom. A “fused ring” is a polycyclic ring system in which two rings share two adjacent atoms, referred to as “bridgehead atoms,” i.e., the two rings share one covalent bond such that the bridgehead atoms are directly connected. A “bridged ring” is a polycyclic ring system in which two rings share three or more atoms separating the bridgehead atoms by a bridge containing at least one atom. Examples of cycloalkyl groups include, but are not limited to, cyclopropyl, cyclobutyl, cyclopentyl, cyclohexyl, cycloheptyl, cyclooctyl, etc.

The term “aryl” as used herein is a group that contains any carbon-based aromatic group including, but not limited to, phenyl, naphthyl, anthracenyl, phenanthranyl, and the like. Aryl moieties are well known and described, for example, in Lewis, R. J., ed., Hawley's Condensed Chemical Dictionary, 13th Edition, John Wiley & Sons, Inc., New York (1997). An aryl group can be substituted or unsubstituted with one or more suitable substituents. An aryl group can comprise a single ring structure (i.e., monocyclic) or multiple ring structures (i.e., polycyclic, e.g., bicyclic or tricyclic). For example, an aryl group can be a monocyclic aryl group, e.g., phenyl.

The term “heterocyclyl” includes stable monocyclic and polycyclic hydrocarbons that contain at least one heteroatom ring member, such as sulfur, oxygen, or nitrogen, wherein the ring structure is saturated or partially unsaturated, provided the ring system is not fully aromatic. A heterocyclyl group can be unsubstituted, or substituted with one or more suitable substituents at any one or more of the carbon atom(s) and/or nitrogen heteroatom(s) of the heterocyclyl. A heterocyclyl can comprise a single ring structure (i.e., monocyclic) or multiple ring structures (i.e., polycyclic, e.g., bicyclic). Polycyclic heterocyclyls include bridged, fused, and spiro ring structures in which at least one ring atom of at least one of the rings of the polycyclic ring system is a heteroatom, for instance oxygen, nitrogen, or sulfur, wherein bridged, fused, and spiro rings are as defined above. A heterocyclyl ring can be attached to the parent molecule at any suitable heteroatom (typically nitrogen) or carbon atom of the ring. The term “4- to 9-membered monocyclic or bicyclic heterocyclyl” includes any four, five, six, seven, eight, or nine membered monocyclic or bicyclic ring structure containing at least one heteroatom ring member selected from oxygen, nitrogen, and sulfur, or independently selected from oxygen and nitrogen, optionally containing one to three additional heteroatoms independently selected from oxygen, nitrogen, and sulfur, or independently selected from oxygen and nitrogen, wherein the ring structure is saturated or partially unsaturated, provided the ring structure is not fully aromatic.

In certain embodiments, the term “heterocyclyl” refers to 4-, 5-, 6-, or 7-membered monocyclic groups and 6-, 7-, 8-, or 9-membered bicyclic groups which have at least one heteroatom (O, S, or N) in at least one of the rings, wherein the heteroatom-containing ring(s) typically has 1, 2, or 3 heteroatoms, such as 1 or 2 heteroatoms, independently selected from O, S, and/or N, or independently selected from O and N. When numbers appear in a subscript after the symbol “C”, the subscript defines with more specificity the number of carbon atoms which that particular heterocycly can contain, in addition to the heteroatoms which that particular heterocycly can contain. For example, “C₁ to C₁₀ heterocycl” or “C₁₋₁₀ heterocycl” is intended to include heterocycl groups having 1, 2, 3, 4, 5, 6, 7, 8, 9, and 10 carbon atoms. Additionally, for example, “C₁ to C₈ heterocycly” or “C₁₋₈ heterocycly” denotes a heterocycl having 1, 2, 3, 4, 5, 6, 7, or 8 carbon atoms.

Examples of monocyclic heterocyclyl groups include, but are not limited to azetidinyl, oxetanyl, tetrahydrofuranyl, pyrrolidinyl, imidazolidinyl, pyrazolidinyl, oxazolidinyl, isoxazolidinyl, thiazolidinyl, isothiazolidinyl, dioxolanyl, dithiolanyl, piperidinyl, piperazinyl, dioxanyl, morpholinyl, azepanyl, oxepanyl, oxazepanyl (e.g., 1,4-oxazepanyl, 1,2-oxazepanyl) and the like. Examples of bicyclic heterocyclyl groups include, but are not limited to, 2-aza-bicyclo[2.2.1]heptanyl, 8-aza-bicyclo[3.2.1]octanyl, 2-aza-spiro[3.3]heptanyl, 3-azabicyclo[2.2.2]octanyl, 3-oxa-9-azabicyclo[3.3.1]nonanyl, 2-oxa-5-azabicyclo[2.2.1]heptanyl, 7-oxa-2-azaspiro[3.5]nonanyl, and 5-azaspiro[2.3]hexanyl and the like.

As used herein, the term “heteroaryl” includes stable monocyclic and polycyclic aromatic hydrocarbons that contain at least one heteroatom ring member such as sulfur, oxygen, or nitrogen. A heteroaryl group can be unsubstituted or substituted with one or more suitable substituents. A heteroaryl can comprise a single ring structure (i.e., monocyclic) or multiple ring structures (i.e., polycyclic, e.g., bicyclic or tricyclic). Each ring of a heteroaryl group containing a heteroatom can contain one or two oxygen or sulfur atoms and/or from one to four nitrogen atoms provided that the total number of heteroatoms in each ring is four or less and each ring has at least one carbon atom. Heteroaryl groups which are polycyclic, e.g., bicyclic or tricyclic must include at least one fully aromatic ring, but the other fused ring or rings can be aromatic or non-aromatic. For example, for a bicyclic heteroaryl, the fused rings completing the bicyclic group can contain only carbon atoms and can be saturated, partially saturated, or unsaturated. A heteroaryl can be attached to the parent molecule at any available nitrogen or carbon atom of any ring of the heteroaryl group. In some embodiments, the term “heteroaryl” refers to 5- or 6-membered monocyclic groups and 9- or 10-membered bicyclic groups which have at least one heteroatom (O, S, or N) in at least one of the rings, wherein the heteroatom-containing ring typically has 1, 2, or 3 heteroatoms, such as 1 or 2 heteroatoms, selected from O, S, and/or N. A heteroaryl group can be unsubstituted, or substituted with one or more suitable substituents at any one or more of the carbon atom(s) and/or nitrogen heteroatom(s) of the heteroaryl. The nitrogen and sulfur heteroatom(s) of a heteroaryl can optionally be oxidized (i.e., N—O and S(O)r, wherein r is 0, 1 or 2).

When numbers appear in a subscript after the symbol “C”, the subscript defines with more specificity the number of carbon atoms which that particular heteroaryl can contain, in addition to the heteroatoms which that particular heteraryl can contain. For example, “C₁ to C₁₀ heteroaryl” or “C₁₋₁₀ heteroaryl” is intended to include heteroaryl groups having 1, 2, 3, 4, 5, 6, 7, 8, 9, and 10 carbon atoms. Additionally, for example, “C₁ to C₈ heteroaryl” or “C₁₋₈ heteroaryl” denotes a heteroaryl having 1, 2, 3, 4, 5, 6, 7, or 8 carbon atoms.

Exemplary monocyclic heteroaryl groups include, but are not limited to, pyrrolyl, pyrazolyl, pyrazolinyl, imidazolyl, oxazolyl, isoxazolyl, thiazolyl, thiadiazolyl, isothiazolyl, furanyl, thiophenyl, oxadiazolyl, pyridinyl, pyrazinyl, pyrimidinyl, pyridazinyl, and triazinyl. Exemplary bicyclic heteroaryl groups include, but are not limited to, indolyl, benzothiazolyl, benzodioxolyl, benzoxazolyl, benzothienyl, quinolinyl, tetrahydroisoquinolinyl, isoquinolinyl, benzimidazolyl, benzopyranyl, indolizinyl, benzofuranyl, chromonyl, coumarinyl, benzopyranyl, cinnolinyl, quinoxalinyl, indazolyl, pyrrolopyridinyl, furopyridinyl, dihydroisoindolyl, and tetrahydroquinolinyl.

The term “alkoxy” as used herein refers to an —O-alkyl group, wherein alkyl is as defined above. An alkoxy group is attached to the parent molecule through a bond to an oxygen atom. An alkoxy group can have a specified number of carbon atoms. For example, “C₁ to C₁₀ alkoxy” or “C₁₋₁₀ alkoxy” is intended to include alkoxy groups having 1, 2, 3, 4, 5, 6, 7, 8, 9, and 10 carbon atoms. Additionally, for example, “C₁ to C₄ alkoxy” or “C₁₋₄ alkoxy” denotes an alkoxy having 1, 2, 3, or 4 carbon atoms. Examples of alkoxy include, but are not limited to, methoxy, ethoxy, propoxy (e.g., n-propoxy, isopropoxy), butoxy (e.g., n-butoxy, isobutoxy, tert-butoxy), pentyloxy (e.g., n-pentyloxy, isopentyloxy, neopentyloxy), etc. An alkoxy group can be unsubstituted or substituted with one or more suitable substituents. Similarly, “alkylthio” or “thioalkoxy” represents an alkyl group as defined above attached to the parent molecule through a bond to a sulfur atom, for example, —S-methyl, —S-ethyl, etc. Representative examples of alkylthio include, but are not limited to, —SCH₃, —SCH₂CH₃, etc.

As used herein, the term “halogen” means fluorine, chlorine, bromine, or iodine. Correspondingly, the term “halo” means fluoro, chloro, bromo, and iodo.

“Haloalkyl” is intended to include both branched and straight-chain saturated aliphatic hydrocarbon radicals substituted with one or more halogen atoms. “Fluorinated alkyl” or “fluoroalkyl” in particular refers to any alkyl group as defined above substituted with at least one fluoro atom, e.g., one to three fluoro atoms, such as one, two, or three fluoroatoms. Examples of haloalkyl include, but are not limited to, fluoromethyl, difluoromethyl, trifluoromethyl, chloromethyl, dichloromethyl, trichloromethyl, pentafluoroethyl, pentachloroethyl, 2,2,2-trifluoroethyl, heptafluoropropyl, and heptachloropropyl. Suitable examples of fluoroalkyl in particular include, but are not limited to, —CF₃, —CHF₂, —CH₂CF₃, —CF₂CF₃, and the like.

The terms “hydroxy” and “hydroxyl” can be used interchangeably, and refer to —OH.

The term “carboxy” and “carboxyl” can be used interchangeably, and refers to —COOH.

The term “ester” refers to —COOR, wherein R is alkyl as defined above.

The term “cyano” refers to —CN.

The term “oxo” refers to a double bonded oxygen group, i.e., a substituent group of the formula ═O.

The term “keto” refers to —C(O)R, wherein R is alkyl as defined above.

As used herein, the term “amino” refers to —NH₂. One or more hydrogen atoms of an amino group can be replaced by a substituent such as an alkyl group, which is referred to as an “alkylamino.” Alkylamino groups have one or both hydrogen atoms of an amino group replaced with an alkyl group and is attached to the parent molecule through a bond to the nitrogen atom of the alkylamino group. For example, alkylamino includes methylamino (—NHCH₃), dimethylamino (—N(CH₃)₂), —NHCH₂CH₃ and the like.

The term “aminoalkyl” as used herein is intended to include both branched and straight-chain saturated aliphatic hydrocarbon groups substituted with one or more amino groups. For example, “C₁₋₄ aminoalkyl” is intended to include alkyl groups having 1, 2, 3, or 4 carbon atoms substituted with one or more amino groups. Aminoalkyl groups are attached to the parent molecule through a bond to a carbon atom of the alkyl moiety of the aminoalkyl group.

Representative examples of aminoalkyl groups include, but are not limited to, —CH₂NH₂, —CH₂CH₂NH₂, and —CH₂CH(NH₂)CH₃.

As used herein, “amido” refers to —C(O)N(R)₂, wherein each R is independently an alkyl group (including both branched and straight-chain alkyl groups) or a hydrogen atom. Examples of amido groups include, but are not limited to, —C(O)NH₂, —C(O)NHCH₃, and —C(O)N(CH₃)₂.

The terms “hydroxyl-substituted alkyl,” “hydroxylalkyl” and “hydroxyalkyl” are used interchangeably, and refer to a branched or straight-chain aliphatic hydrocarbon group substituted with one or more hydroxyl groups. Hydroxyalkyl groups are attached to the parent molecule through a bond to a carbon atom of the alkyl moiety of the hydroxyalkyl group. A hydroxyalkyl group can have a specified number of carbon atoms. For example, “C₁ to C₁₀ hydroxyalkyl” or “C₁₋₁₀ hydroxyalkyl” is intended to include hydroxyalkyl groups having 1, 2, 3, 4, 5, 6, 7, 8, 9, and 10 carbon atoms. Additionally, for example, “C₁ to C₄ hydroxylalkyl” or “C₁₋₄ hydroxyalkyl” denotes a hydroxyalkyl group having 1, 2, 3, or 4 carbon atoms. Examples of hydroxyalkyl include, but are not limited to, hydroxylmethyl (—CH₂OH), hydroxylethyl (—CH₂CH₂OH), etc.

As used herein, “amide” refers to —N(R′)C(O)R, wherein each R and R′ is independently chosen from hydrogen, alkyl, cycloalkyl, aryl, and heteroaryl. Examples of amide groups include, but are not limited to, —NHC(O)CH₃, —NHC(O)CH₂CH₃, and —N(CH₃)C(O)CH₃.

As used herein, “carbamide” refers to —N(R′)C(O)N(R)₂, wherein each R and R′ is independently chosen from hydrogen, alkyl, cycloalkyl, aryl, and heteroaryl. Examples of carbamide groups include, but are not limited to, —NHC(O)NH₂, —NHC(O)NHCH₃ (methyl carbamide), and —NHC(O)NH(Ph).

As used herein, “sulfonamide” refers to —N(R′)SO₂—R, wherein each R and R′ is independently chosen from hydrogen, alkyl, cycloalkyl, aryl, and heteroaryl. Examples of sulfonamide groups include, but are not limited to, —NHSO₂CH₃ (methyl sulfonamide), and —NH SO₂Ph.

In accordance with convention used in the art:

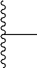

is used in structural formulas herein to depict the bond that is the point of attachment of a group, moiety or substituent to the core, backbone, or parent molecule structure.

When a bond to a substituent is shown to cross a bond connecting two atoms in a ring, then such substituent can be bonded to any atom on the ring.

The term “substituted” as used herein with respect to any organic radical (e.g., alkyl, cycloalkyl, heteroaryl, aryl, heterocyclyl, etc.) means that at least one hydrogen atom is replaced with a non-hydrogen group, provided that all normal valencies are maintained and that the substitution results in a stable compound. When a particular group is “substituted,” that group can have one or more substituents, such as from one to five substituents, one to three substituents, or one to two substituents, independently selected from the list of substituents. The term “independently” when used in reference to substituents, means that when more than one of such substituents is possible, such substituents can be the same or different from each other. Examples of suitable substituents include, but are not limited to, alkyl, halo, haloalkyl, alkoxy, amido, hydroxy, hydroxyalkyl, amino, carboxyl, ester, oxo, cyano and the like.

When any variable occurs more than one time in any constituent or formula for a compound, its definition at each occurrence is independent of its definition at every other occurrence. Thus, for example, if a group is shown to be substituted with 0-3 R groups, then said group can be optionally substituted with up to three R groups, and at each occurrence, R is selected independently from the definition of R.

The terms “optional” or “optionally” mean that the event or circumstance described can, but need not, occur, and such a description includes the situation in which the event or circumstance does or does not occur. For example, “optionally substituted heterocyclyl” means that a substituent group can be, but need not be, present, and such a description includes the situation of the heterocyclyl group being substituted by a suitable substituent and the heterocyclyl group not being substituted by any substituent.

One skilled in the art will recognize that in certain embodiments compounds described herein can have one or more asymmetric carbon atoms in their structure. As used herein, any chemical formulas with bonds shown only as solid lines and not as solid wedged or hashed wedged bonds, or otherwise indicated as having a particular configuration (e.g., R or S) around one or more atoms, contemplates each possible stereoisomer, or mixture of two or more stereoisomers. Stereoisomers includes enantiomers and diastereomers. Enantiomers are stereoisomers that are non-super-imposable mirror images of each other. A 1:1 mixture of a pair of enantiomers is a racemate or racemic mixture. Diastereomers (or dia-stereoisomers) are stereoisomers that are not enantiomers, i.e., they are not related as mirror images, and occur when two or more stereoisomers of a compound have different configurations at one or more of the equivalent stereocenters and are not mirror images of each other. Substituent groups (e.g., alkyl, heterocyclyl, etc.) can contain stereocenters in either the R or S configuration.

Certain examples contain chemical structures that comprise (R) or (S) terminology. When (R) or (S) is used in the name of a compound or in the chemical representation of the compound, it is intended to mean that the compound is a single isomer at that stereocenter, with established absolute configuration of either (R) or (S).

Stereochemically pure isomeric forms can be obtained by techniques known in the art in view of the present disclosure. For example, diastereoisomers can be separated by physical separation methods such as fractional crystallization and chromatographic techniques, and enantiomers can be separated from each other by the selective crystallization of the diastereomeric salts with optically active acids or bases or by chiral chromatography. Pure stereoisomers can also be prepared synthetically from appropriate stereochemically pure starting materials, or by using stereoselective reactions.

Compounds described herein can also form tautomers. The term “tautomer” refers to compounds that are interchangeable forms of a particular compound structure and that vary in the displacement of hydrogen atoms and electrons. Tautomers are constitutional isomers of chemical compounds that readily interconvert, usually resulting in relocation of a proton (hydrogen). Thus, two structures can be in equilibrium through the movement of pi electrons and an atom (usually hydrogen). All tautomeric forms and mixtures of tautomers of the compounds described herein are included with the scope of the present disclosure.

Compounds described herein can exist in solvated and unsolvated forms. The term “solvate” means a physical association, e.g., by hydrogen bonding, of a compound described herein with one or more solvent molecules. The solvent molecules in the solvate can be present in a regular arrangement and/or a non-ordered arrangement. The solvate can comprise either a stoichiometric or nonstoichiometric amount of the solvent molecules. “Solvate” encompasses both solution-phase and isolable solvates. Compounds described herein can form solvates with water (i.e., hydrates) or common organic solvents. Exemplary solvates include, but are not limited to, hydrates, ethanolates, methanolates, and isopropanolates. Methods of solvation are generally known in the art.

Also included within the scope of the present disclosure are all isotopes of atoms occurring in the compounds described herein, including intermediates and final products. Isotopes include those atoms having the same atomic number but different mass numbers. By way of general example and without limitation, isotopes of hydrogen include deuterium and tritium. Isotopes of carbon include ¹³C and ¹⁴C.

The present disclosure further includes isotopically-labeled compounds. An “isotopically-labeled” or “radio-labeled” compound is a compound of the present disclosure where one or more atoms are replaced or substituted by an atom having an atomic mass or mass number different from the atomic mass or mass number typically found in nature (i.e., naturally occurring). Isotopically-labeled compounds can generally be prepared by conventional techniques known to those skilled in the art or by processes analogous to those described herein, using an appropriate isotopically-labeled reagent in place of the non-labeled reagent otherwise employed.

As used herein, the name of a compound is intended to encompass all possible existing isomeric forms, including stereoisomers (e.g., enantiomers, diastereomers, racemate or racemic mixture, and any mixture thereof) of the compound.

### Selective Alpha-2C Adrenergic Receptor (ADRA2C) Agonists

In one general aspect, the present disclosure relates to a selective alpha-2C adrenergic receptor (ADRA2C) agonist, which is useful for treating or preventing a disease, such as one of TNF-α-mediated inflammatory and autoimmune diseases.

As used herein, the term “selective alpha-2C adrenergic receptor agonist” or “selective ADRA2C agonist” refers to a compound that specifically binds to and activates alpha-2C subtype of adrenergic receptors, which are part of the adrenergic receptor family. These receptors are G protein-coupled receptors involved in the sympathetic nervous system's regulation of various physiological processes. These agonists are designed to selectively target the alpha-2C receptor, thereby minimizing interactions with alpha-2A receptor. Examples of selective ADRA2C agonist include, but not limited to, the compounds described herein, such as the compounds in Table 1.

Any methods known in the filed can be used to characterize a compound's selective binding activity to alpha-2C receptor over alpha-2A receptor. For example, one experimental measure of the selectivity is effect selectivity ratio (ESR).

As used herein, “ESR” is defined as the ratio of the EC50 value for alpha-2A receptor to the EC50 value for alpha-2C receptor. It is calculated as follows:

$$ESR = EC {50}_{alpha - 2 A} / EC {50}_{alpha - 2 C} .$$

A compound can be considered as a “selective ADRA2C agonist” if its ESR is higher than 5, 10, 20, 40, 100, 200, 400, or 800.

In some embodiments, the selective ADRA2C agonist is an alpha-2A adrenergic receptor (ADRA2A) antagonist. As used herein, the term “alpha-2A adrenergic receptor antagonist” or “ADRA2A antagonist” refers to a compound that specifically binds to and block or inhibits the alpha-2A subtype of adrenergic receptors. According to embodiment of the present disclosure, the Ki value for the alpha-2A antagonist and alpha-2A receptor is lower than 80 nM, 40 nM, 20 nM, 10 nM, 5 nM, 2 nM, or 1 nM.

In certain embodiments, the alpha-2A antagonist has a low alpha-2A agonist activity. Preferably, the alpha-2A antagonist's EC50 value is higher than 100 nM, 200 nM, 400 nM, 800 nM, 1600 nM, 3200 nM, 5000 nM, or 10000 nM.

In certain embodiments, the alpha-2A adrenergic receptor antagonist is an alpha-2A partial agonist. As used herein, the term “alphs-2A partial agonist” refers to a compound that binds and activates the alpha-2A adrenergic receptor but with lower efficacy than a full alpha-2A agonist. Accordingly, the alpha-2A partial agonist acts as a functional antagonist in the presence of a full agonist, since it competes for the receptor but produces a weaker response.

In certain embodiments, the alpha-2A adrenergic receptor antagonist has an intrinsic activity (IA) value lower than 60%, 20%, or 10%. As used herein, the term “intrinsic activity” or “IA” refers to a drug's ability to activate a receptor after binding to it. It determines how strong of a response a drug can produce when it interacts with a receptor. Intrinsic activity of a test agonist is defined as:

$$IA = maximal response to the test agonist / maximal response to full agonist .$$

In some embodiments, the selective ADRA2C agonist is peripherally restrictive. As used herein, the term “peripherally restrictive” means that a compound has its primary mechanism of action outside of the central nervous system (CNS), usually because it is excluded from the CNS by the blood-CNS barrier.

In some embodiments, the selective ADRA2C agonist is non-peripherally restrictive. As used herein, the term “non-peripherally restrictive” means that a compound can be readily distributed into the CNS after being administered into a subject, bind to and activate α2AR receptor in both the central nervous system (brain and spinal cord) and the peripheral nervous system.

In some embodiments, the selective ADRA2C agonist is a compound of formula (I):

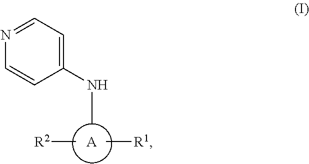

or a stereoisomer, tautomer, pharmaceutically acceptable salt or a solvate thereof,
wherein,
ring A is a bicyclic ring;
R¹ is chosen from —C₀₋₁₂ haloalkyl, —C(O)—C₀₋₁₂ alkyl, —C(O)—C₀₋₁₂ haloalkyl, —C(O)-aryl, —C(O)-heteroaryl, —C₀₋₁₂ alkylene-COOH, —C(O)—NR³R^{3′}, —CN, —NR³R^{3′}, —NC(O)R³R^{3′}, —NR³—SO₂—R^{3′}, —O—C₀₋₁₂ alkyl, —SO₂—R³, —SO₂—NR³R^{3′}, —C₀₋₁₂ alkylene-C₂₋₁₂ heteroaryl, and —C₀₋₁₂ alkylene-C₂₋₁₂ heterocyclyl;
R² is chosen from hydrogen, halogen, haloalkyl, and alkyl; and
each of R³ and R^{3′} is independently hydrogen, haloalkyl, or alkyl.

In some embodiments, the compound of formula (I) is a compound of formula (I-1):

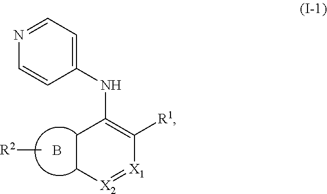

wherein,
X₁ is N or CH;
X₂ is N or CH; and
ring B is aryl or heteroaryl.

In certain embodiments, X₁ and X₂ are CH, or X₁ and X₂ are N.

In certain embodiments, X is X₁ is CH and X₂ is N, or X₁ is N and X₂ is CH.

In certain embodiments, ring B is an aryl, particularly C₁₋₁₂ aryl such as phenyl.

In certain embodiments, ring B is a heteroaryl, particularly C₂₋₁₂ heteroaryl, such as pyridine, furan, or thiophene. In certain embodiments, ring B is a thiophene.

In some embodiments, ring A is

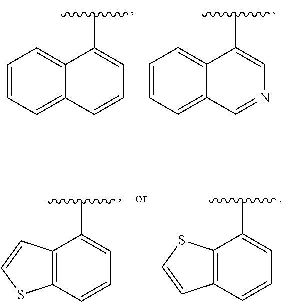

In certain embodiments, ring A is

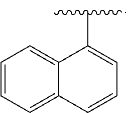 [SMILES: *c1cccc2ccccc12]

In certain embodiments, ring A is

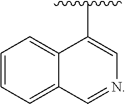 [SMILES: *c1cncc2ccccc12]

In certain embodiments, ring A is

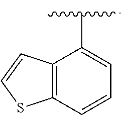 [SMILES: *c1cccc2sccc12]

In certain embodiments, ring A is

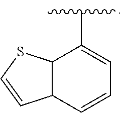 [SMILES: *C1=CC=CC2C=CSC12]

In some embodiments, R¹ is —C₀₋₆ haloalkyl, —C(O)—C₀₋₆ alkyl, —C₀₋₆ alkylene-COOH, —NR^{3′}—SO₂—R³, —SO₂—R³, or —C₂₋₁₂ heterocyclyl. In some embodiments, R¹ is —C(O)—C₀₋₆ haloalkyl, —C(O)-aryl, —C(O)-heteroaryl, —CN, —NR³R^{3′}, —O—C₀₋₆ alkyl, or —C₂₋₁₂ heteroaryl.

In certain embodiments, R¹ is —C(O)-Me, —COOH, —NH—SO₂-Me, —SO₂-Me, or

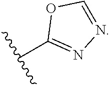 [SMILES: *c1nnco1]

In certain embodiments, R¹ is —CF₂(Me), —O-Me, —C(O)—CF₃, —CN, —C(O)-phenyl, —SO₂-Me, —N(CF₃)-Me,

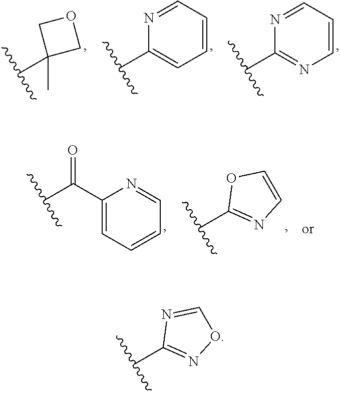

In certain embodiments, R¹ is —CF₂(Me), —O-Me, —C(O)—CF₃, —CN, —SO₂-Me, or —N(CF₃)-Me. In certain embodiments, R¹ is or

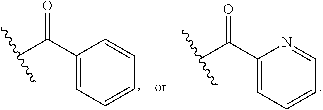 [SMILES: *C(=O)c1ccccc1 ; *C(=O)c1ccccn1]

In certain embodiments, R² is

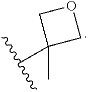 [SMILES: *C1(C)COC1]

In certain embodiments, R¹ is

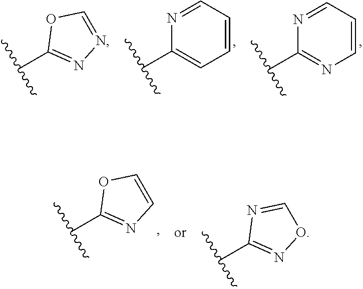

In some embodiments, R² is hydrogen. In some embodiments, R² is halogen, such as fluoride, chloride, or bromide. In certain embodiments, R² is chloride.

Exemplary compounds of formula (I) or (I-1) include, but are not limited to, the compounds described herein, and any tautomer, stereoisomer, pharmaceutically acceptable salt or solvate thereof.

In particular embodiments, provided is a compound selected from Compounds 1-24, or a tautomer, stereoisomer, pharmaceutically acceptable salt or solvate thereof.

All possible combinations of the above-indicated embodiments of compounds of formula (I) or (I-1) and their tautomers, stereoisomers, pharmaceutically acceptable salts and solvates are considered to be embraced within the scope of the present disclosure.

Exemplary compounds of formula (I) or (I-1) include, but are not limited to, the following compounds, and any tautomer, stereoisomer, pharmaceutically acceptable salt or solvate thereof:

TABLE 1

| Compound No. | Compound |
|---|---|
| 1 | 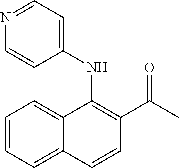 [SMILES: CC(=O)c1ccc2ccccc2c1Nc1ccncc1] |
| 2 | 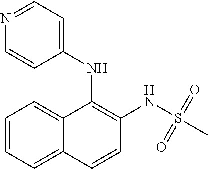 [SMILES: CS(=O)(=O)Nc1ccc2ccccc2c1Nc1ccncc1] |
| 3 | 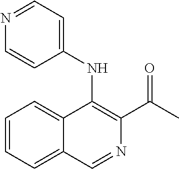 [SMILES: CC(=O)c1ncc2ccccc2c1Nc1ccncc1] |
| 4 | 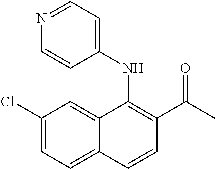 [SMILES: CC(=O)c1ccc2ccc(Cl)cc2c1Nc1ccncc1] |
| 5 | 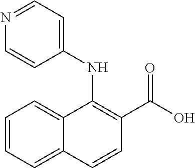 [SMILES: O=C(O)c1ccc2ccccc2c1Nc1ccncc1] |
| 6 | 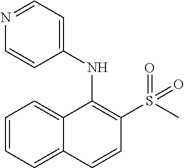 [SMILES: CS(=O)(=O)c1ccc2ccccc2c1Nc1ccncc1] |
| 7 | 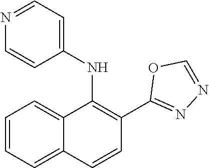 [SMILES: c1ccc2c(Nc3ccncc3)c(-c3nnco3)ccc2c1] |
| 8 | 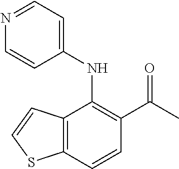 [SMILES: CC(=O)c1ccc2sccc2c1Nc1ccncc1] |
| 9 | 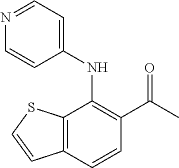 [SMILES: CC(=O)c1ccc2ccsc2c1Nc1ccncc1] |
| 10 | 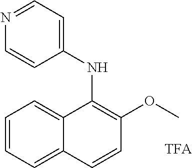 [SMILES: COc1ccc2ccccc2c1Nc1ccncc1] |
| 11 | 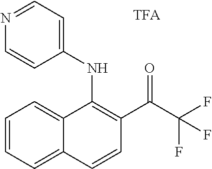 [SMILES: O=C(c1ccc2ccccc2c1Nc1ccncc1)C(F)(F)F] |
| 12 | 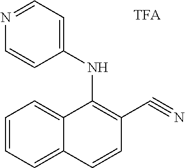 [SMILES: N#Cc1ccc2ccccc2c1Nc1ccncc1] |
| 13 | 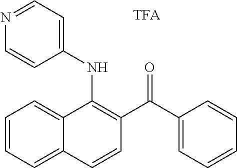 [SMILES: O=C(c1ccccc1)c1ccc2ccccc2c1Nc1ccncc1] |
| 14 | 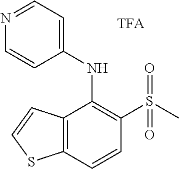 [SMILES: CS(=O)(=O)c1ccc2sccc2c1Nc1ccncc1] |
| 15 | 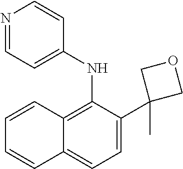 [SMILES: CC1(c2ccc3ccccc3c2Nc2ccncc2)COC1] |
| 16 | 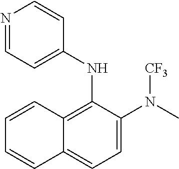 [SMILES: CN(c1ccc2ccccc2c1Nc1ccncc1)C(F)(F)F] |
| 17 | 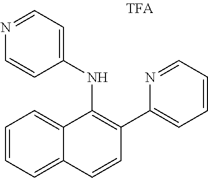 [SMILES: c1ccc(-c2ccc3ccccc3c2Nc2ccncc2)nc1] |
| 18 | 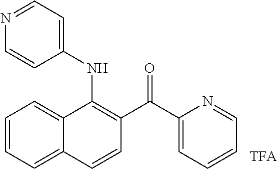 [SMILES: O=C(c1ccccn1)c1ccc2ccccc2c1Nc1ccncc1] |
| 19 | 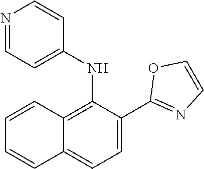 [SMILES: c1ccc2c(Nc3ccncc3)c(-c3ncco3)ccc2c1] |
| 20 | 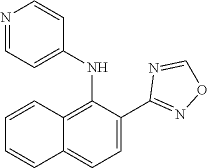 [SMILES: c1ccc2c(Nc3ccncc3)c(-c3ncon3)ccc2c1] |
| 21 | 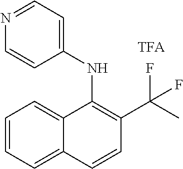 [SMILES: CC(F)(F)c1ccc2ccccc2c1Nc1ccncc1] |
| 22 | 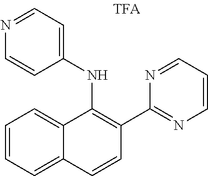 [SMILES: c1cnc(-c2ccc3ccccc3c2Nc2ccncc2)nc1] |
| 23 | 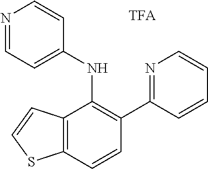 [SMILES: c1ccc(-c2ccc3sccc3c2Nc2ccncc2)nc1] |
| 24 | 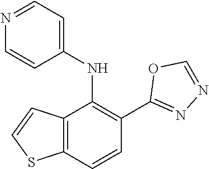 [SMILES: c1cc(Nc2c(-c3nnco3)ccc3sccc23)ccn1] |

### Methods of Preparation

Compounds described herein can be prepared by any number of processes that is known in the field and more specifically illustrated by the exemplary compounds which follow in the Examples section herein. The compounds provided herein as prepared in the processes described below can be synthesized in the form of mixtures of stereoisomers (e.g., enantiomers, diastereomers), including racemic mixtures of enantiomers, which can be separated from one another using art-known resolution procedures, for instance including liquid chromatography using a chiral stationary phase. Additionally or alternatively, stereochemically pure isomeric forms of the compounds described herein can be derived from the corresponding stereochemically pure isomeric forms of the appropriate starting materials, intermediates, or reagents. For example, if a specific stereoisomer is desired, the compound can be synthesized by stereospecific methods of preparation, which typically employ stereochemically pure starting materials or intermediate compounds.

Pharmaceutically acceptable salts of compounds described herein can be synthesized from the parent compound containing an acidic or basic moiety by conventional chemical methods. Generally, such salts can be prepared by reacting the free acid or base forms of these compounds with a stoichiometric amount of the appropriate acid or base in water or in an organic solvent, or in a mixture of the two. Examples of suitable organic solvents include, but are not limited to, ether, ethyl acetate (EtOAc), ethanol, isopropanol, or acetonitrile.

By way of illustration, but not as a limitation, compounds of formula (I) or (I-1) described herein can be prepared according to the examples shown in the present disclosure. One of ordinary skill in the art will recognize that, to obtain various compounds of formula (I) or (I-1) as described herein, starting materials can be suitably selected so that the ultimately desired substituent groups will be carried through (i.e., be stable over the course of the synthesis) the reaction scheme with or without protection as appropriate to yield the desired product. Alternatively, it may be necessary or desirable to employ, in place of the ultimately desired substituent, a suitable group that may be carried through (i.e., be stable over the course of the synthesis) the reaction scheme and replaced as appropriate with the desired substituent.

If no temperature or temperature range is stated, it is to be understood that the reaction is to be conducted at room temperature.

When isomerically pure samples are desired, isomeric mixtures of compounds synthesized can be separated by chiral supercritical fluid chromatography (SFC) or high performance liquid chromatography (HPLC).

### Compositions

In one aspect, provided is a pharmaceutical composition comprising a compound of formula (I) or (I-1) or a stereoisomer, tautomer, pharmaceutically acceptable salt or solvate thereof, as described herein.

Compositions can also comprise a pharmaceutically acceptable carrier. A pharmaceutically acceptable carrier is non-toxic and should not interfere with the efficacy of the active ingredient. Pharmaceutically acceptable carriers can include one or more excipients such as binders, disintegrants, swelling agents, suspending agents, emulsifying agents, wetting agents, lubricants, flavorants, sweeteners, preservatives, dyes, solubilizers and coatings. The precise nature of the carrier or other material can depend on the route of administration, e.g., intramuscular, intradermal, subcutaneous, oral, intravenous, cutaneous, intramucosal (e.g., gut), intranasal or intraperitoneal routes. For liquid injectable preparations, for example, suspensions and solutions, suitable carriers and additives include water, glycols, oils, alcohols, preservatives, coloring agents and the like. For solid oral preparations, for example, powders, capsules, caplets, gelcaps and tablets, suitable carriers and additives include starches, sugars, diluents, granulating agents, lubricants, binders, disintegrating agents and the like. For nasal sprays/inhalant mixtures, the aqueous solution/suspension can comprise water, glycols, oils, emollients, stabilizers, wetting agents, preservatives, aromatics, flavors, and the like as suitable carriers and additives.

Compositions can be formulated in any matter suitable for administration to a subject to facilitate administration and improve efficacy, including, but not limited to, oral (enteral) administration and parenteral injections. The parenteral injections include intravenous injection or infusion, subcutaneous injection, intradermal injection, and intramuscular injection. Compositions can also be formulated for other routes of administration including transmucosal, ocular, rectal, long acting implantation, sublingual administration, under the tongue, from oral mucosa bypassing the portal circulation, inhalation, or intranasal.

The pharmaceutical compositions are suitable for clinical and veterinary use, including mammals such as human, dogs, cats, cattle, horses, pigs, sheep and goats.

The form of the pharmaceutical compositions, the route of administration, the dosage and the regimen depend upon the condition to be treated, such as the severity of the illness, the age, weight, and sex of the patient. Pharmaceutical compositions can be formulated for different modes of administration such as for topical, oral, intranasal, parenteral, intraocular, intravenous, intramuscular, or subcutaneous administration.

In yet another aspect, provided is a method of preparing a pharmaceutical composition comprising combining a compound of formula (I) or (I-1) or a stereoisomer, tautomer, pharmaceutically acceptable salt or solvate thereof, with at least one pharmaceutically acceptable carrier. Pharmaceutical compositions can be prepared by any method known in the art in view of the present disclosure, and one of ordinary skill in the art will be familiar with such techniques used to prepare pharmaceutical compositions. For example, a pharmaceutical composition according to the present disclosure can be prepared by mixing a compound of formula (I) or (I-1) with one or more pharmaceutically acceptable carriers according to conventional pharmaceutical compounding techniques, including but not limited to, conventional admixing, dissolving, granulating, emulsifying, encapsulating, entrapping or lyophilizing processes.

### Methods of Use

In one general aspect, provided are methods of selectively activating alpha-2C adrenergic receptor and methods of treating or preventing a disease in a subject, using an effective amount of a selective alpha-2C adrenergic receptor (ADRA2C) agonist, such as the compounds described herein or the composition containing the compounds with one or more acceptable pharmaceutical carriers, describe herein.

As used herein, “an effective amount” means an amount of a composition or compound that elicits a biological or medicinal response in a tissue system or subject that is being sought by a researcher, veterinarian, medical doctor or other conditions, which can include alleviation of the symptoms of the disease, disorder, or condition being treated. An effective amount can vary depending upon a variety of factors, such as the physical condition of the subject, age, weight, health, etc.; and the particular disease, disorder, or condition to be treated. An effective amount can readily be determined by one of ordinary skill in the art in view of the present disclosure.

In some embodiments, the compounds of formula (I) or (I-1) can be useful for selectively activating alpha-2C adrenergic receptor.

In some embodiments, provided is a method of selectively activating alpha-2C adrenergic receptor in a subject in need thereof, comprising administering to the subject a compound or composition described herein, e.g., administering an effective amount of a compound or composition described herein.

In some embodiments, provided is a method of treating or preventing a disease in human or in mammal animal, such as dog, cat, cattle, horse, pig, sheep and goat.

In some embodiments, provided is a method of treating or preventing a disease in a subject in need thereof, comprising administering to the subject a compound or composition described herein, e.g., administering an effective amount of a compound or composition described herein.

In some embodiments, the disease is nasal congestion, chronic rhinitis, neuropathic pain, inflammatory pain, Raynaud's phenomenon, hypotensive shock, cognitive impairment and neurodegenerative diseases (e.g., Alzheimer's disease, Parkinson's disease, Huntington's disease, amyotrophic lateral sclerosis (ALS), multiple sclerosis (MS), ADHD), schizophrenia, alcohol and drug withdrawal syndrome, rosacea, overactive bladder (OAB), glaucoma, potential tumor growth modulation, etc.

In some embodiments, the selective ADRA2C agonist has an ESR (alpha-2A/alpha-2C) higher than 5, 10, 20, 40, 100, 200, 400, or 800.

In some embodiments, the selective ADRA2C agonist is an alpha-2A adrenergic receptor (ADRA2A) antagonist.

In certain embodiments, the alpha-2A antagonist has a low alpha-2A agonist activity. Preferably, the alpha-2A antagonist's EC50 value is higher than 100 nM, 200 nM, 400 nM, 800 nM, 1600 nM, 3200 nM, 5000 nM, or 10000 nM.

In certain embodiments, the alpha-2A adrenergic receptor antagonist is an alpha-2A partial agonist.

In certain embodiments, the alpha-2A adrenergic receptor antagonist has an intrinsic activity (IA) value lower than 60%, 20%, or 10%.

In some embodiments, the selective ADRA2C agonist is peripherally restrictive.

In some embodiments, the selective ADRA2C agonist is non-peripherally restrictive.

In some embodiments, the selective ADRA2C agonist is a compound of formula (I) as described herein.

In some embodiments, the selective ADRA2C agonist is a compound chosen from Table 1.

In some embodiments, the compounds and pharmaceutical compositions described herein cause less side effects, such as sedation, bradycardia, hypotension, and hypothermia in the treated subject.

In certain embodiments, the compounds and pharmaceutical compositions described herein do not cause sedative response in the treated subject.

In another one general aspect, provided are methods of down-regulating TNF-α in a subject in need thereof and methods of treating or preventing one of TNF-α-mediated inflammatory and autoimmune diseases in a subject, using selective alpha-2C adrenergic receptor (ADRA2C) agonist, such as the compounds described herein or the composition containing the compounds with one or more acceptable pharmaceutical carriers, describe herein.

In some embodiments, the subject is a human.

In some embodiments, the subject is a mammal animal, such as dog, cat, cattle, horse, pig, sheep and goat.

In some embodiments, provided is a method of down-regulating TNF-α in a subject in need thereof, comprising administering to the subject a compound or composition described herein, e.g., administering an effective amount of a compound or composition described herein.

In certain embodiments, the method down-regulate the expression, production and secretion of TNF-α in the subject.

In some embodiments, provided is a method of treating or preventing one of TNF-α-mediated inflammatory and autoimmune diseases in human or in mammal animal.

In some embodiments, provided is a method of treating or preventing one of TNF-α-mediated inflammatory and autoimmune diseases in a subject in need thereof, comprising administering to the subject a compound or composition described herein, e.g., administering an effective amount of a compound or composition described herein.

As used herein, “TNF-α-mediated inflammatory and autoimmune diseases” refer to various immune-mediated inflammatory diseases and autoimmune disorders that are driven by TNF-α as a critical pro-inflammatory cytokine. Examples of the diseases include, but are not limited to, rheumatoid arthritis, inflammatory bowel disease (IBDS), Crohn's disease, psoriasis, ankylosing spondylitis, sepsis and septic inflammation, acute lung injury (ALI) and acute respiratory distress syndrome (ARDS).

In some embodiments, the compound or composition described herein can significantly inhibit LPS-induced inflammatory responses and effectively reduce the expression, production and secretion of TNF-α.

In some embodiments, the compound or composition described can have the advantages of clear mechanism, high activity, good stability, low cost and potential for oral administration, and are suitable for both human and veterinary uses.

### Examples

The following examples are to further illustrate the nature of the present disclosure. It should be understood that the following examples do not limit the disclosure and the scope of the present disclosure is to be determined by the appended claims.

### Methods of Synthesis

Unless indicated otherwise, the abbreviations for chemical reagents and synthesis conditions have their ordinary meaning known in the art as follows:
“ACN” refers to acetonitrile;
“LDA” refers to lithium diisopropyl amide;
“EA” or “EtOAc” refers to ethyl acetate;
“PE” refers to petroleum ether;
“r.t.” and “rt” refer to room temperature;
“THF” refers to tetrahydrofuran;
“DIPEA” refers to diisopropylethylamine;
“DCM” refers to dichloromethane;
“HOBT” refers to hydroxybenzotriazole;
“TLC” refers to thin layer chromatography;
“DMF” refers to dimethylformamide;
“h” refers to hours;
“min” refers to minutes;
“EDCI” refers to 1-Ethyl-3-(3-dimethylaminopropyl)carbodiimide;
“DMAP” refers to 4-Dimethylaminopyridine;
“Prep-HPLC” refers to preparative high performance liquid chromatography;
“DPPF” refers to 1,1′-Bis(diphenylphosphino)ferrocene; and
“NCS” refers to N-chlorosuccinimide.
“TEA” refers to triethylamine.
“TES” refers to triethyl silane.
“Trt” refers to trityl group or triphenylmethyl group.
“MeOH” refers to methanol.
“EtOH” refers to ethanol.
“t-BuXphos” refers to tert-butyl-Xantphos
“TMAl” refers to trimethylaluminum
“Xantphos” refers to 9,9-dimethyl-4,5-bis(diphenylphosphino)xanthene
“Pd(PPH₃)₄” refers to tetrakis(triphenylphosphine)palladium(0)

### Example 1. Synthesis of Compound 1

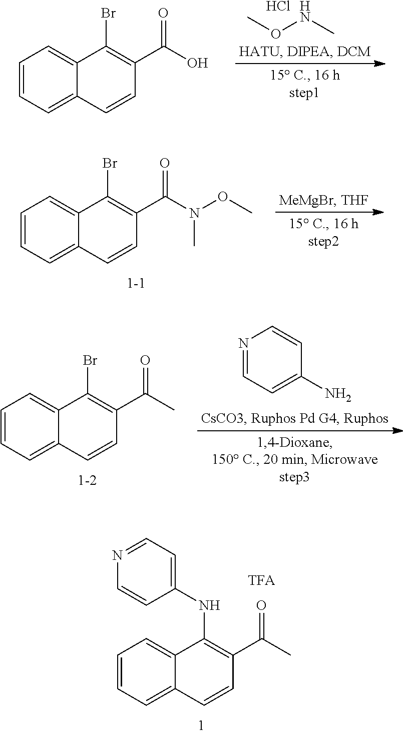

Step 1: A solution of 1-bromo-2-naphthoic acid (1.00 g, 3.98 mmol, 1.0 eq) in dichloromethane (10 mL) was treated with N-methoxymethanamine hydrochloride (777 mg, 7.97 mmol, 2.0 eq), DIPEA (2.06 g, 15.92 mmol, 4.0 eq), and HATU (1.82 g, 4.78 mmol, 1.2 eq) under a nitrogen atmosphere. The reaction mixture was stirred at 25° C. for 16 h. Upon completion, as confirmed by LC-MS, purification by flash column chromatography on silica gel afforded Compound 1-1 (1.05 g) as a solid. Yield: 89.7%.

Step 2: A solution of Compound 1-1 (1.00 g, 3.41 mmol, 1.0 eq) in tetrahydrofuran (10 mL) was cooled to −78° C., followed by the addition of a 3.0 M solution of methylmagnesium bromide in THF. The reaction mixture was stirred at 25° C. for 16 h. Upon completion, as confirmed by LC-MS, a saturated ammonium chloride solution was added, and the mixture was extracted with ethyl acetate. The combined organic layers were dried over Na₂SO₄, filtered, and concentrated under vacuum. Purification by silica gel column chromatography yielded Compound 1-2 (431 mg) as a white solid. Yield: 50.9%.

Step 3: A solution of Compound 1-2 (200 mg, 0.807 mmol, 1.0 eq) in 1,4-dioxane (4 mL) was combined with 4-aminopyridine (91.1 mg, 0.96 mmol, 1.2 eq), Cs₂CO₃ (657.3 mg, 2.02 mmol, 2.5 eq), RuPhos Pd G4 (34.3 mg, 0.04 mmol, 0.05 eq), and RuPhos (37.7 mg, 0.081 mmol, 0.1 eq) under a nitrogen atmosphere. The reaction mixture was stirred at 100° C. for 16 h. LC-MS analysis indicated a large amount of unreacted starting material. Additional RuPhos Pd G4 (102 mg, 0.12 mmol, 0.15 eq) and RuPhos (114 mg, 0.244 mmol, 0.3 eq) were introduced, and the reaction was continued at 150° C. for 20 min under microwave irradiation. Upon completion, as confirmed by LC-MS, the reaction mixture was filtered and concentrated under vacuum. The residue was dissolved in dichloromethane (2 mL) and trifluoroacetic acid (1 mL) and stirred at 10° C. for 2 min. The mixture was then concentrated under vacuum, and the residue was purified by preparative HPLC to afford Compound 1 (59 mg) as a white solid. Yield: 27.8%.

### Example 2. Synthesis of Compound 2

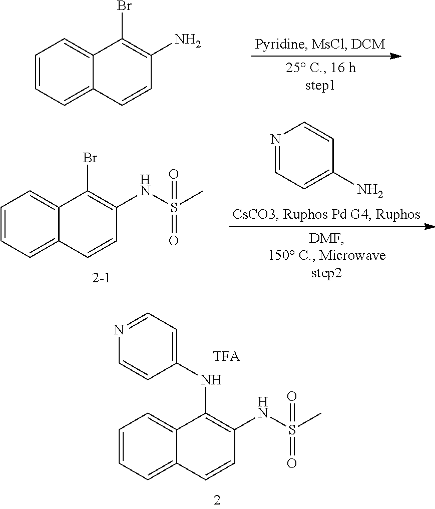 [SMILES: Nc1ccc2ccccc2c1Br ; CS(=O)(=O)Nc1ccc2ccccc2c1Br ; Nc1ccncc1 ; CS(=O)(=O)Nc1ccc2ccccc2c1Nc1ccncc1]

Step 1: A solution of 1-bromo-2-naphthylamine (500 mg, 2.25 mmol, 1.0 eq) in dichloromethane (5 mL) was treated with pyridine (214 mg, 2.70 mmol, 1.2 eq) and methanesulfonyl chloride (MsCl) (283.5 mg, 2.48 mmol, 1.1 eq) under a nitrogen atmosphere. The reaction mixture was stirred at 25° C. for 3.5 h. LC-MS analysis indicated the presence of unreacted starting material. Additional MsCl (129 mg, 1.13 mmol, 0.5 eq) was introduced under nitrogen, and stirring was continued at 25° C. for 0.5 h. Upon completion, the mixture was purified by flash column chromatography on silica gel to afford Compound 2-1 (438 mg) as a solid. Yield: 65.1%.

Step 2: A solution of Compound 2-1 (50 mg, 0.167 mmol, 1.0 eq) in DMF (2 mL) was combined with 4-aminopyridine (19 mg, 0.202 mmol, 1.2 eq), Cs₂CO₃ (136 mg, 0.418 mmol, 2.5 eq), RuPhos Pd G4 (14.2 mg, 0.0167 mmol, 0.1 eq), and RuPhos (15.6 mg, 0.0334 mmol, 0.2 eq) under a nitrogen atmosphere. The reaction was heated to 160° C. in a microwave reactor for 2.5 h. LC-MS analysis showed a significant amount of unreacted starting material. Additional Cs₂CO₃ (136 mg, 0.418 mmol, 2.5 eq), RuPhos Pd G4 (36 mg, 0.042 mmol, 0.25 eq), and RuPhos (40 mg, 0.086 mmol, 0.5 eq) were added, and the reaction was further heated at 180° C. in a microwave reactor for 20 min. Upon completion, as confirmed by LC-MS, the reaction mixture was filtered and concentrated under vacuum. The residue was dissolved in dichloromethane (1 mL) and trifluoroacetic acid (0.5 mL) and stirred at 10° C. for 2 min. The mixture was then concentrated under vacuum, and the residue was purified by preparative HPLC to afford Compound 2 (4.4 mg) as a white solid. Yield: 6.2%.

Overall yield=4.03%.

### Example 3. Synthesis of Compound 5

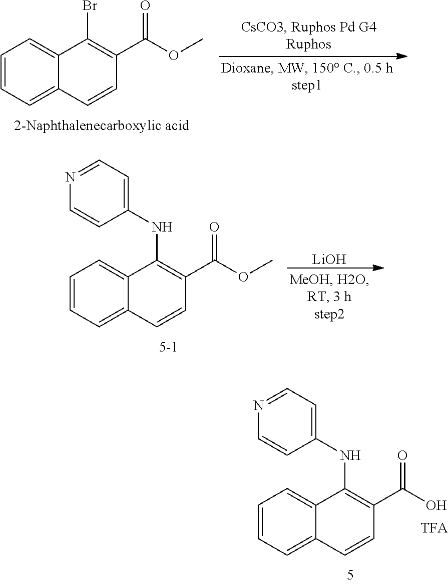

Step 1: A solution of methyl 1-bromo-2-naphthalenecarboxylate (500 mg, 1.89 mmol, 1.0 eq) in 1,4-Dioxane was combined with 4-Aminopyridine (213 mg, 2.26 mmol, 1.2 eq), Cs₂O₃ (1.536 g, 4.72 mmol, 2.5 eq), Ruphos Pd G4 (80 mg, 0.189 mmol, 0.05 eq), and Ruphos (88 mg, 0.189 mmol, 0.1 eq) under a nitrogen atmosphere. The reaction mixture was stirred for 20 min at 150° C. in a microwave reactor. LC-MS confirmed reaction completion. The mixture was filtered and concentrated under vacuum, and the crude product was purified by silica gel column chromatography (MeOH/DCM=1:20) to afford Compound 5-1 (150 mg) as a white solid. Yield: 28.6%.

Step 2: Compound 5-1 (150 mg, 0.54 mmol, 1.0 eq) and LiOH (68 mg, 1.62 mmol, 3.0 eq) were stirred in a mixture of MeOH and water for 3 h at room temperature under a nitrogen atmosphere. LC-MS confirmed reaction completion. The reaction mixture was evaporated to dryness, and the crude product was purified by preparative HPLC to afford Compound 5 (41.2 mg) as a white solid. Yield: 28.8%.

Overall yield=8.25%.

### Example 4. Synthesis of Compound 6

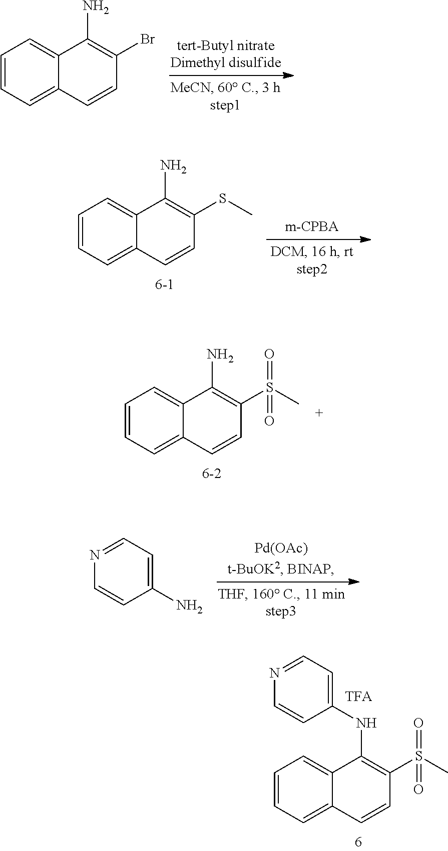 [SMILES: Nc1c(Br)ccc2ccccc12 ; CSc1ccc2ccccc2c1N ; CS(=O)(=O)c1ccc2ccccc2c1N ; Nc1ccncc1 ; CS(=O)(=O)c1ccc2ccccc2c1Nc1ccncc1]

Step 1: A mixture of 2-amino-1-bromonaphthalene (500 mg, 2.25 mmol, 1.0 eq) and dimethyl disulfide (846 mg, 9.0 mmol, 4.0 eq) in MeCN (30 mL) was heated to 60° C. under a nitrogen atmosphere. Tert-butyl nitrite (348 mg, 3.38 mmol, 1.5 eq) was then added dropwise, and the reaction mixture was stirred for 3 h at 60° C. LC-MS confirmed reaction completion. The mixture was poured into water, washed with EA, dried over Na₂SO₄, and purified by fast silica gel column chromatography. Another reaction using 2-amino-1-bromonaphthalene (400 mg) was combined, and the product was purified to afford Compound 6-1 (300 mg) as a faint yellow oil. Yield: 29.13%.

Step 2: Compound 6-1 (300 mg, 1.18 mmol, 1.0 eq) in DCM (6 mL) was cooled to 0° C. under a nitrogen atmosphere. m-CPBA (408 mg, 2.36 mmol, 2.0 eq) was added, and the reaction mixture was stirred for 16 h at room temperature. Another portion of m-CPBA (408 mg, 2.36 mmol, 2.0 eq) was then added, and stirring was continued for another 16 h at room temperature. LC-MS confirmed reaction completion. The mixture was filtered through a Celite pad, and the filtrate was evaporated to dryness. The residue was purified by fast silica gel column chromatography to afford Compound 6-2 (360 mg) as a faint yellow solid, which was used in the next step without further purification.

Step 3: Compound 6-2 (100 mg, 0.35 mmol, 1.0 eq), 4-aminopyridine (33 mg, 0.35 mmol, 1.0 eq), t-BuOK (118 mg, 1.05 mmol, 3.0 eq), Pd(OAc)₂ (9 mg, 0.04 mmol, 0.1 eq), and BINAP (25 mg, 0.04 mmol, 0.1 eq) were added to THF (2 mL) in a 10 mL vial, and the system was purged with N₂. The reaction mixture was heated at 160° C. in a microwave reactor for 11 min. LC-MS confirmed reaction completion. The mixture was poured into water, washed with EA, dried over Na₂SO₄, and treated with TFA (0.5 mL). The solvent was evaporated to dryness, and the crude product was purified by fast silica gel column chromatography and preparative HPLC to afford Compound 6 (44.6 mg) as a faint yellow solid. Yield: 28.83%.

Overall yield=8.40%.

### Example 5. Synthesis of Compound 7

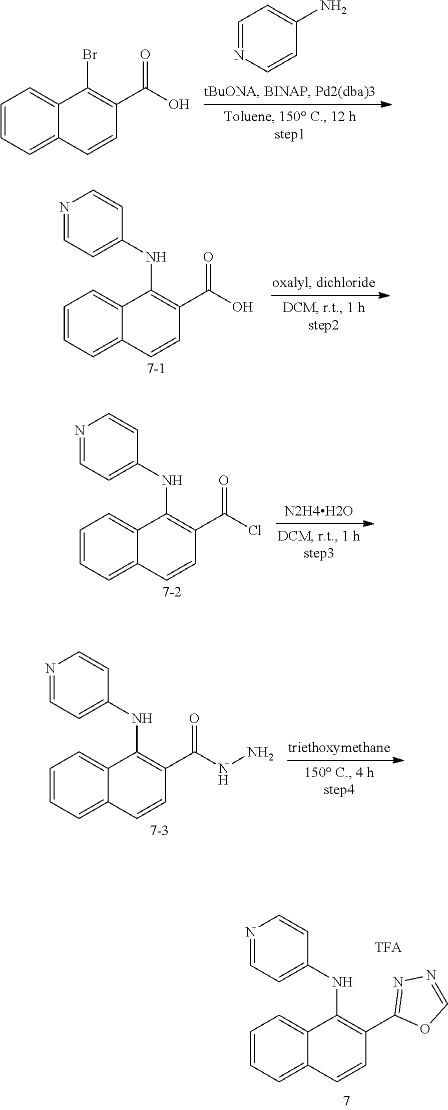

Step 1: A solution of 1-bromo-2-naphthoic acid (2.0 g, 7.97 mmol, 1.0 eq) in toluene (30 mL) was treated with pyridin-4-amine (899 mg, 9.56 mmol, 1.2 eq), t-BuONa (1.2 g, 12.74 mmol, 1.6 eq), Pd₂(dba)₃ (364 mg, 0.39 mmol, 0.05 eq), and BINAP (496 mg, 0.79 mmol, 0.1 eq). The reaction mixture was stirred at 150° C. for 12 hours under a nitrogen atmosphere. Completion of the reaction was confirmed by LC-MS. The mixture was filtered, and the filter cake was suspended in DCM:MeOH=10:1 (100 mL). The suspension was adjusted to pH=8 with 1 M HCl, followed by extraction with DCM (3×30 mL). The aqueous layer was concentrated under reduced pressure to afford Compound 7-1 (500 mg) as a yellow oil. Yield: 23.7% Step 2: A solution of Compound 7-1 (500 mg, 1.89 mmol, 1.0 eq) in DCM (10 mL) was treated with DMF (0.1 mL) and oxalyl chloride (0.5 mL, 5.68 mmol, 3.0 eq) at 0° C. The mixture was stirred at 15° C. for 1 hour, and completion of the reaction was confirmed by TLC. The organic phase was concentrated under reduced pressure to give Compound 7-2 (500 mg) as a yellow oil, which was used directly in the next step without further purification. Yield: 100% Step 3: A solution of Compound 7-2 (500 mg) in DCM (5 mL) was treated with hydrazine hydrate (volume not specified) at 0° C., and the mixture was stirred at 15° C. for 1 hour. Completion of the reaction was confirmed by LC-MS. The mixture was concentrated under reduced pressure, and the crude residue was purified by preparative HPLC, affording Compound 7-3 (60 mg) as a white solid. Yield: 11.4%

Step 4: A solution of Compound 7-3 (60 mg) in triethoxymethane (0.6 mL) was stirred at 150° C. for 4 hours. Completion of the reaction was confirmed by LC-MS. The organic layer was purified by preparative HPLC, affording Compound 7 (20 mg) as a white solid. Yield: 23.8% Overall yield: 0.64%

### Example 6. Synthesis of Compound 8

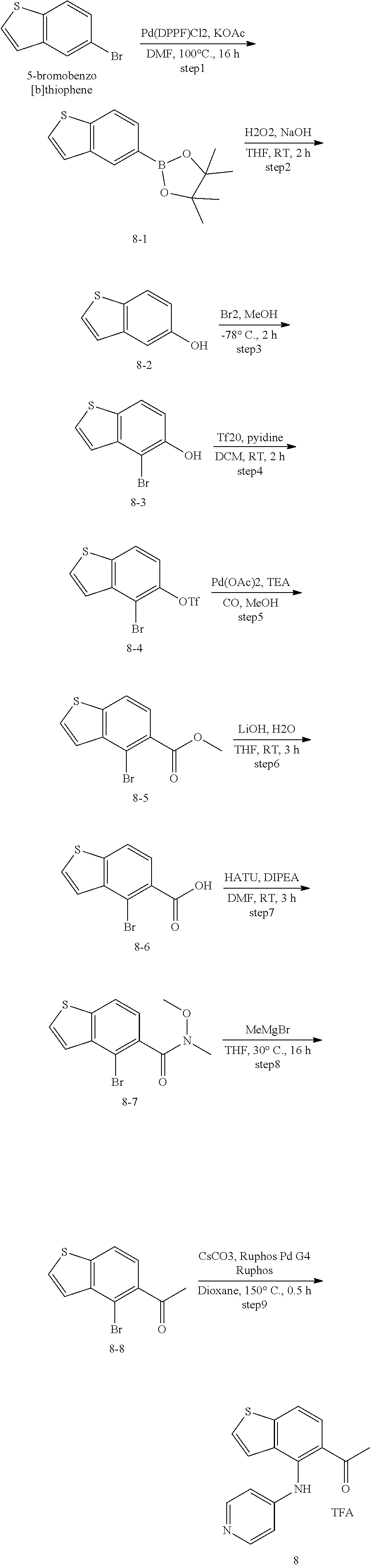

Step 1: A suspension of 5-bromobenzo[b]thiophene (25 g, 0.12 mol, 1.0 eq), bis(pinacolato)diboron (89.4 g, 0.35 mol, 3.0 eq), potassium acetate (57 g, 0.59 mol, 5.0 eq), and Pd(DPPF)Cl₂ (2 g, 0.0023 mol, 0.02 eq) in DMF was stirred at 100° C. for 16 hours under a nitrogen atmosphere. Completion of the reaction was confirmed by LC-MS. The mixture was filtered through a Celite pad, and the filtrate was diluted with water (1 L), followed by extraction with ethyl acetate (2×500 mL). The combined organic layers were washed with brine, dried over anhydrous Na₂SO₄, filtered, and concentrated under reduced pressure. The residue was purified by silica gel column chromatography (eluent: PE/EA=50:1), affording Compound 8-1 (34 g) as a white solid. Yield: 100%

Step 2: A solution of Compound 8-1 (34 g, 0.13 mol, 1.0 eq) in THF was treated with NaOH (1 N, 119 mL, 3.5 vol) and hydrogen peroxide (34 mL, 1.0 vol). The reaction was stirred at room temperature for 2 hours. Completion was confirmed by LC-MS. The mixture was diluted with saturated sodium thiosulfate, extracted with ethyl acetate, and the organic layers were washed with brine, dried over anhydrous Na₂SO₄, filtered, and concentrated under reduced pressure. The residue was purified by silica gel chromatography (eluent: PE/EA=20:1) to afford Compound 8-2 (10 g) as a colorless oil. Yield: 56.8%

Step 3: A suspension of Compound 8-2 (10 g, 0.066 mol, 1.0 eq) and NBS (12.4 g, 0.07 mol, 1.05 eq) in DCM was stirred at 10° C. for 1 hour under nitrogen. Completion was confirmed by LC-MS. The mixture was filtered through Celite, diluted with water (200 mL), and extracted with ethyl acetate (100 mL). The organic layer was washed, dried, filtered, and concentrated under reduced pressure. The residue was purified by silica gel chromatography (eluent: PE/EA=9:1), affording Compound 8-3 (11 g) as a colorless oil. Yield: 73%

Step 4: A solution of Compound 8-3 (1.0 g, 4.4 mmol, 1.0 eq) in DCM was cooled to −10° C., followed by addition of pyridine (0.52 g, 6.5 mmol, 9.6 eq) and trifluoroacetic anhydride (0.52 g, 6.5 mmol, 1.5 eq). The reaction mixture was stirred at room temperature for 2 hours under nitrogen. Completion was confirmed by LC-MS. After dilution with water (20 mL) and extraction with ethyl acetate (2×10 mL), the organic layers were dried, filtered, and concentrated under reduced pressure. The residue was purified by silica gel chromatography (eluent: PE/EA=9:1), affording Compound 8-4 (1.6 g) as a white solid. Yield: 100%

Step 5: A mixture of Compound 8-4 (1.6 g, 4.4 mmol, 1.0 eq), TEA (0.85 g, 8.4 mmol, 1.9 eq), DPPP (0.44 g, 1.1 mmol, 0.24 eq), and Pd(OAc)₂ (0.20 g, 0.89 mmol, 0.2 eq) in DMSO/MeOH (1:1, 32 mL total) was stirred at 80° C. for 8 hours under nitrogen. Completion was confirmed by LC-MS. The mixture was diluted with water (100 mL), extracted with ethyl acetate (2×50 mL), and the combined organic layers were dried, filtered, and concentrated. The residue was purified by silica gel chromatography (eluent: PE/EA=10:1), affording Compound 8-5 (0.5 g) as a white solid. Yield: 41.6%

Step 6: A mixture of Compound 8-5 (500 mg, 1.84 mmol, 1.0 eq) and LiOH (232 mg, 5.53 mmol, 3.0 eq) in MeOH/H₂O was stirred at 26° C. for 2 hours under nitrogen. Completion was confirmed by LC-MS. The reaction mixture was concentrated, then poured into water and acidified to pH 1. The product was extracted with DCM/MeOH (10:1), and the organic layers were dried, filtered, and concentrated under reduced pressure, yielding Compound 8-6 (420 mg) as a crude product, which was used directly in the next step. Yield: 89%

Step 7: A solution of Compound 8-6 (420 mg, 1.63 mmol, 1.0 eq) in DMF was treated with N,O-dimethylhydroxylamine hydrochloride (239 mg, 2.45 mmol, 1.5 eq), HATU (745 mg, 1.96 mmol, 1.2 eq), and DIPEA (1056 mg, 8.17 mmol, 5.0 eq) at room temperature. The mixture was stirred for 16 hours under nitrogen. Completion was confirmed by LC-MS. After dilution with water (20 mL) and extraction with ethyl acetate (2×20 mL), the organic layers were dried, filtered, and concentrated. The crude was purified by silica gel chromatography (eluent: PE/EA=1:1), affording Compound 8-7 (490 mg) as a white solid.

Yield: 100%

Step 8: A solution of Compound 8-7 (490 mg, 1.63 mmol, 1.0 eq) in THF was cooled to −10° C., followed by addition of methylmagnesium bromide (0.65 mL, 1.96 mmol, 1.2 eq). The reaction mixture was stirred at room temperature for 2 hours under nitrogen. Completion was confirmed by LC-MS. After dilution with water (20 mL) and extraction with ethyl acetate (2×10 mL), the organic layers were dried, filtered, and concentrated. The crude was purified by silica gel chromatography (eluent: PE/EA=9:1), affording Compound 8-8 (220 mg) as a white solid. Yield: 52.8%

Step 9: A mixture of Compound 8-8 (180 mg, 0.71 mmol, 1.0 eq), 4-aminopyridine (80 mg, 0.85 mmol, 1.2 eq), Cs₂CO₃ (575 mg, 1.76 mmol, 2.5 eq), RuPhos Pd G4 (30 mg, 0.035 mmol, 0.05 eq), and RuPhos (33 mg, 0.071 mmol, 0.1 eq) in 1,4-dioxane was stirred at 150° C. for 40 minutes under microwave irradiation. Completion was confirmed by LC-MS. The mixture was filtered, concentrated, and the crude residue was purified by preparative HPLC, affording Compound 8 (87 mg) as a white solid. Yield: 46%

Overall yield: 3.7%

### Example 7. Synthesis of Compound 9

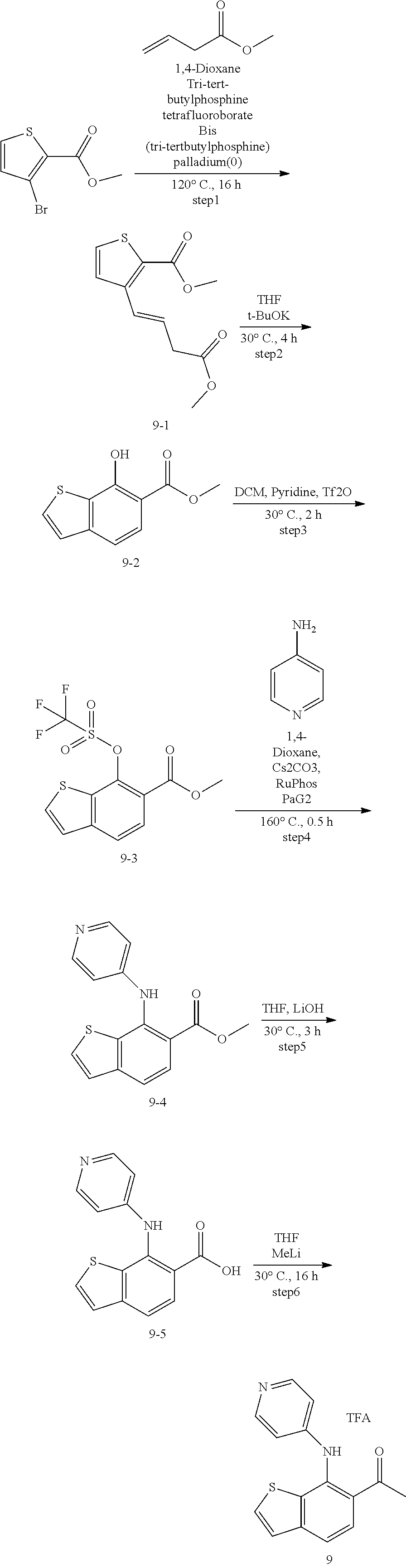

Step 1: A mixture containing methyl 3-bromothiophene-2-carboxylate (5.0 g, 23.0 mmol, 1.0 eq), methyl 3-butenoate (3.4 g, 34.0 mmol, 1.5 eq), and N-cyclohexyl-N-methylcyclohexanamine (9.8 g, 50.6 mmol, 2.2 eq) in 1,4-dioxane (50 mL) was stirred at 50° C. for 6 minutes under a nitrogen atmosphere. Tri-tert-butylphosphine tetrafluoroborate (200 mg, 0.69 mmol, 0.03 eq) was then added and the mixture was stirred for an additional 6 minutes at 50° C. Bis(tri-tert-butylphosphine)palladium(0) (117 mg, 0.23 mmol, 0.01 eq) was subsequently added, and the resulting mixture was stirred at 120° C. for 16 hours under nitrogen. Reaction completion was confirmed by LC-MS. After filtration and concentration under reduced pressure, the crude residue was purified by silica gel column chromatography, yielding Compound 9-1 (5.5 g). Yield: 99.6% Step 2: A reaction mixture consisting of 9-1 (5.0 g, 21.0 mmol, 1.0 eq) in THF (100 mL) was cooled to 5° C. under nitrogen. Potassium tert-butoxide (3.3 g, 29.0 mmol, 1.4 eq) was added, and the mixture was stirred at 30° C. for 4 hours. Reaction completion was confirmed by LC-MS. The mixture was diluted with 2 N HCl and extracted with ethyl acetate (2×20 mL). The combined organic layers were dried over anhydrous sodium sulfate, filtered, and concentrated under vacuum. Purification by flash chromatography afforded Compound 9-2 (1.6 g). Yield: 38% Step 3: A reaction mixture consisting of Compound 9-2 (1.6 g, 8.0 mmol, 1.0 eq) and pyridine (1.9 g, 24.0 mmol, 3.0 eq) in DCM (20 mL) was cooled to 5° C. under nitrogen. Trifluoromethanesulfonic anhydride (2.9 g, 13.6 mmol, 1.7 eq) was added, and the reaction was stirred at 30° C. for 2 hours. Completion was confirmed by LC-MS. The reaction mixture was concentrated under reduced pressure, and the crude product was purified by silica gel column chromatography, affording Compound 9-3 (2.2 g). Yield: 80.9%

Step 4: A mixture of Compound 9-3 (1.0 g, 2.9 mmol, 1.0 eq), 4-aminopyridine (332 mg, 3.5 mmol, 1.2 eq), cesium carbonate (2.4 g, 7.25 mmol, 2.5 eq), RuPhos Palladacycle Gen.4 (123 mg, 0.145 mmol, 0.05 eq), and dicyclohexyl[2-[2,6-di(propan-2-yloxy)phenyl]phenyl]phosphine (135 mg, 0.29 mmol, 0.1 eq) in 1,4-dioxane (20 mL) was heated at 160° C. for 30 minutes using microwave irradiation. Completion of the reaction was confirmed by LC-MS. The reaction mixture was filtered and concentrated under vacuum. The residue was purified by silica gel column chromatography, yielding Compound 9-4 (540 mg). Yield: 65.5%

Step 5: A mixture of Compound 9-4 (540 mg, 1.9 mmol, 1.0 eq), lithium hydroxide (239 mg, 5.7 mmol, 3.0 eq), and water (5 mL) in THF (10 mL) was stirred at 30° C. for 3 hours under a nitrogen atmosphere. Completion was confirmed by LC-MS. The mixture was concentrated under reduced pressure to give a crude residue. Then, 1 N HCl (5.7 mL) was added, and the resulting mixture was filtered to yield Compound 9-5 (300 mg). Yield: 58.5%

Step 6: A solution of Compound 9-5 (100 mg, 0.37 mmol, 1.0 eq) in THF (1 mL) was cooled to 0° C. under nitrogen, followed by the addition of methyl lithium (1.5 mL, 1.5 mmol, 4.0 eq). The reaction mixture was stirred at 30° C. for 16 hours. Incomplete reaction was confirmed by LC-MS. The reaction mixture was diluted with water and extracted with ethyl acetate (2×20 mL). The organic phase was dried over anhydrous sodium sulfate, filtered, and concentrated under vacuum. The crude residue was purified by preparative HPLC, affording Compound 9 (10 mg).

Yield: 10%

Overall yield: 1.17%

### Example 8. Synthesis of Compound 11

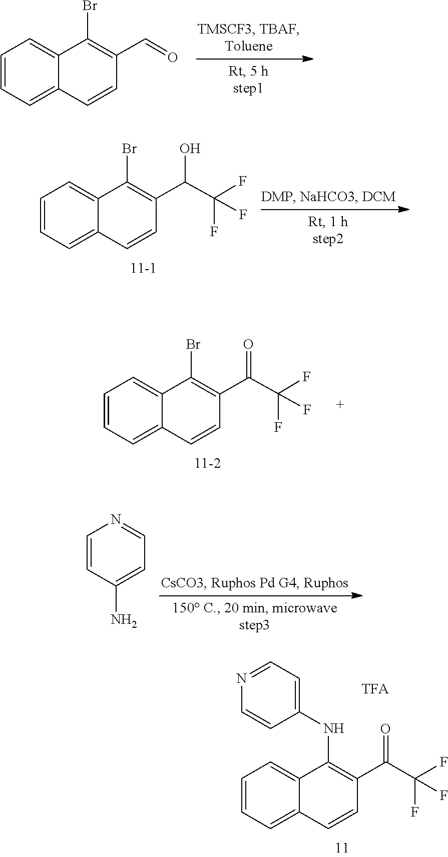 [SMILES: O=Cc1ccc2ccccc2c1Br ; OC(c1ccc2ccccc2c1Br)C(F)(F)F ; O=C(c1ccc2ccccc2c1Br)C(F)(F)F ; Nc1ccncc1 ; O=C(c1ccc2ccccc2c1Nc1ccncc1)C(F)(F)F]

Step 1: A solution of 1-bromo-2-naphthaldehyde (500 mg, 2.13 mmol, 1.0 eq) in toluene (5 mL) was treated with TMSCF₃ (378.6 mg, 2.66 mmol, 1.25 eq) and TBAF (56 mg, 0.213 mmol, 0.1 eq) under a nitrogen atmosphere. The mixture was stirred at 20° C. for 1 hour, and reaction completion was confirmed by LC-MS. The mixture was diluted with water and extracted with ethyl acetate. The organic phase was dried over anhydrous sodium sulfate and concentrated under reduced pressure. The crude residue was purified by flash chromatography, affording Compound 11-1 (295 mg). Yield: 45.56%

Step 2: A solution of Compound 11-1 (295 mg, 0.97 mmol, 1.0 eq) in DCM (3 mL) was prepared. Dess-Martin periodinane (988 mg, 2.33 mmol, 2.4 eq) and sodium bicarbonate (326 mg, 3.88 mmol, 4.0 eq) were added, and the mixture was stirred at room temperature for 2 hours. Reaction completion was confirmed by GC-MS. After addition of sodium thiosulfate, saturated aqueous sodium bicarbonate, and diethyl ether, the organic phase was separated and the aqueous layer was extracted with diethyl ether. The combined organic layers were dried over anhydrous sodium sulfate, concentrated under reduced pressure, and purified by column chromatography, affording Compound 11-2 (200 mg) as a white solid. Yield: 68.26%

Step 3: A solution of Compound 11-2 (100 mg, 0.33 mmol, 1.0 eq) in 1,4-dioxane (2 mL) was combined with 4-aminopyridine (37.4 mg, 0.40 mmol, 1.2 eq), cesium carbonate (269 mg, 0.825 mmol, 2.5 eq), RuPhos Pd G4 (35 mg, 0.041 mmol, 0.12 eq), and RuPhos (42 mg, 0.09 mmol, 0.27 eq) under a nitrogen atmosphere. The reaction mixture was heated at 150° C. for 20 minutes using microwave irradiation. GC-MS analysis indicated the presence of unreacted starting material. The mixture was filtered and concentrated under reduced pressure. The resulting crude residue was dissolved in DCM (2 mL) and treated with TFA (1 mL) at 15° C. for 2 minutes. The mixture was then concentrated in vacuo, and the residue was purified by preparative HPLC, yielding Compound 11 (44 mg) as a white solid. Yield: 30.99%

Overall yield: 9.64%

### Example 9. Synthesis of Compound 12

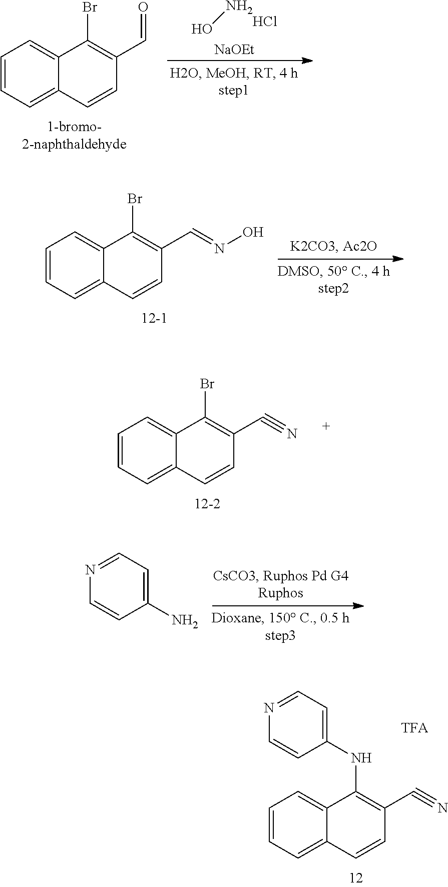

Step 1: A solution of 1-bromo-2-naphthaldehyde (1.00 g, 4.3 mmol, 1.0 eq) in methanol and water was prepared, and hydroxylamine hydrochloride (0.41 g, 5.9 mmol, 1.4 eq) and sodium ethoxide (0.70 g, 8.5 mmol, 2.0 eq) were added under a nitrogen atmosphere. The resulting suspension was stirred at room temperature for 4 hours. Completion of the reaction was confirmed by LC-MS. The mixture was diluted with water and extracted with ethyl acetate. The organic layers were combined, washed with brine, dried over anhydrous sodium sulfate, filtered, and concentrated under reduced pressure. The resulting solid, Compound 12-1 (1.1 g), was obtained as a white solid and used directly in the next step without further purification. Yield: 100%

Step 2: A mixture of Compound 12-1 (1.10 g, 4.4 mmol, 1.0 eq) in DMSO was treated with potassium carbonate (1.21 g, 8.8 mmol, 2.0 eq) and acetic anhydride (0.90 g, 8.8 mmol, 2.0 eq) under nitrogen. The reaction was stirred at 80° C. for 4 hours, and reaction completion was confirmed by LC-MS. The reaction mixture was diluted with water and extracted with ethyl acetate. The combined organic layers were washed with brine, dried over anhydrous sodium sulfate, filtered, and concentrated under reduced pressure. The crude residue was purified by silica gel column chromatography (eluent: petroleum ether/ethyl acetate=10:1), affording Compound 12-2 (750 mg) as a white solid. Yield: 76.5%

Step 3: A solution of Compound 12-2 (100 mg, 0.43 mmol, 1.0 eq) in 1,4-dioxane was combined with 4-aminopyridine (49 mg, 0.52 mmol, 1.2 eq), cesium carbonate (351 mg, 1.08 mmol, 2.5 eq), RuPhos Pd G4 (18 mg, 0.021 mmol, 0.05 eq), and RuPhos (20.1 mg, 0.043 mmol, 0.1 eq) under nitrogen atmosphere. The reaction mixture was stirred at 140° C. for 16 hours, and completion was confirmed by LC-MS. The mixture was filtered, and the filtrate was concentrated under reduced pressure. The crude product was purified by preparative HPLC, affording Compound 12 (74 mg) as a white solid. Yield: 70.5%

Overall yield: 54.0%

### Example 10. Synthesis of Compound 13

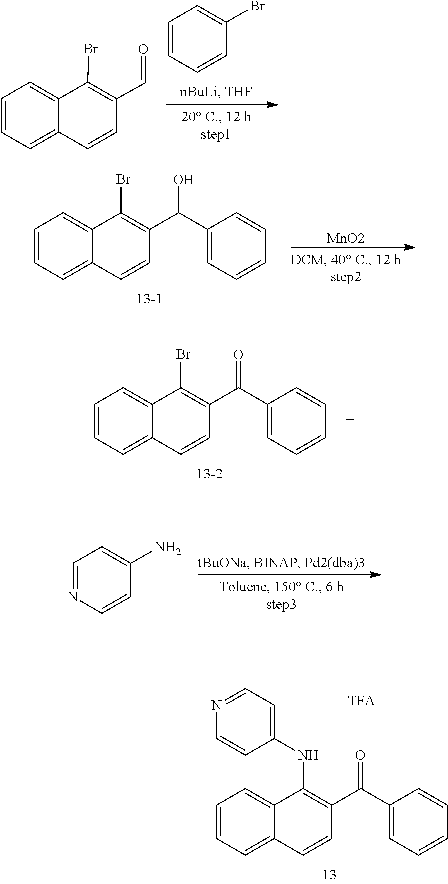

Step 1: A mixture of THF (10 mL) and bromobenzene (1.00 g, 6.38 mmol, 1.5 eq) was added to a 100 mL reaction bottle under nitrogen. The reaction was cooled to −78° C., and n-BuLi (2.55 mL, 6.38 mmol, 1.5 eq, 2.5 M) was added dropwise. After stirring at −78° C. for 1 hour, a solution of 1-bromo-2-naphthaldehyde (1.00 g, 4.25 mmol, 1.0 eq) in THF (10 mL) was added dropwise. The reaction mixture was stirred at 25° C. for 12 hours, and reaction completion was confirmed by LC-MS. Afterward, NH₄Cl (20 mL) was added, and the aqueous phase was extracted with ethyl acetate (3×10 mL). The organic layers were combined, dried over anhydrous sodium sulfate, filtered, and concentrated under reduced pressure. Purification by silica gel column chromatography afforded Compound 13-1 (1.00 g) as a yellow oil. Yield: 76.9%

Step 2: A solution of Compound 13-1 (1.00 g, 3.19 mmol, 1.0 eq) in DCM (20 mL) was treated with MnO₂ (2.78 g, 31.9 mmol, 10 eq). The reaction mixture was stirred at 40° C. for 12 hours, and reaction completion was confirmed by TLC. The mixture was filtered, and the filtrate was concentrated under vacuum. The residue was subjected to silica gel column chromatography, affording Compound 13-2 (900 mg) as a yellow oil. Yield: 90.6%

Step 3: A mixture containing Compound 13-2 (100 mg, 0.32 mmol, 1.0 eq), pyridin-4-amine (36.29 mg, 0.38 mmol, 1.2 eq), t-BuONa (49.41 mg, 0.51 mmol, 1.6 eq), Pd₂(dba)₃ (29.43 mg, 0.032 mmol, 0.1 eq), and BINAP (40.02 mg, 0.064 mmol, 0.2 eq) was stirred in toluene (2 mL) at 150° C. for 6 hours. Reaction completion was confirmed by LC-MS. The reaction mixture was filtered, and the filtrate was concentrated under reduced pressure. The crude product was purified by preparative HPLC, yielding Compound 13 (21 mg) as a white solid. Yield: 15.6%

Overall yield: 10.9%

### Example 11. Synthesis of Compound 17

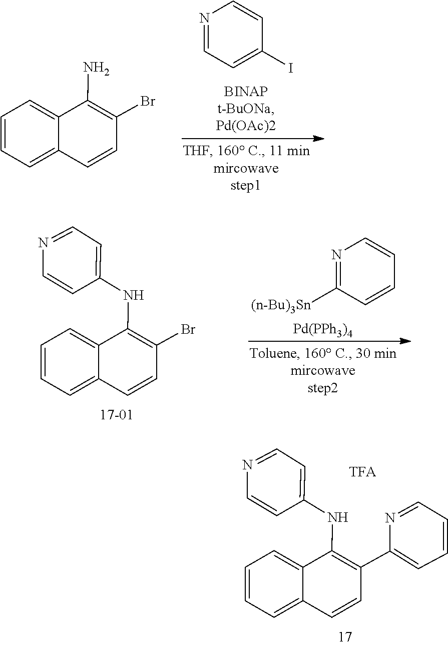

Step 1: To a 30 mL vial was added THF (20 mL), 1-amino-2-bromonaphthalene (0.6 g, 2.7 mmol, 1 eq), 4-iodopyridine (0.558 g, 2.7 mmol, 1 eq), t-BuONa (0.906 g, 9.5 mmol, 3.5 eq), Pd(OAc)₂ (0.072 g, 0.3 mmol, 0.1 eq), and BINAP (0.198 g, 0.3 mmol, 0.1 eq). The system was purged with nitrogen. The reaction mixture was stirred for 11 min at 160° C. under microwave irradiation. LC-MS indicated that the reaction was complete. The mixture was extracted with EA. The organic phase was dried over Na₂SO₄, concentrated to dryness, and purified by fast silica gel column chromatography to give Compound 17-01 (0.5 g) as a brown solid. Yield: 61.93%. [M+H]⁺=299.1/301.1.

Step 2: To a 30 mL vial was added toluene (10 mL), Compound 17-01 (0.2 g, 0.7 mmol, 1 eq), 2-(tri-n-butylstannyl)pyridine (0.3 g, 0.8 mmol, 1.1 eq), and Pd(PPh₃)₄ (0.04 g, 0.035 mmol, 0.05 eq). The system was purged with nitrogen. The reaction mixture was stirred for 30 min at 160° C. under microwave irradiation. LC-MS indicated that the reaction was complete. TFA (0.5 mL) was added to the mixture and the solvent was evaporated to dryness. The residue was purified by preparative TLC followed by preparative HPLC to give Compound 17 (0.085 g) as a yellow solid.

Yield: 29.54%.

Overall yield: 18.29%.

### Example 12. Synthesis of Compound 18

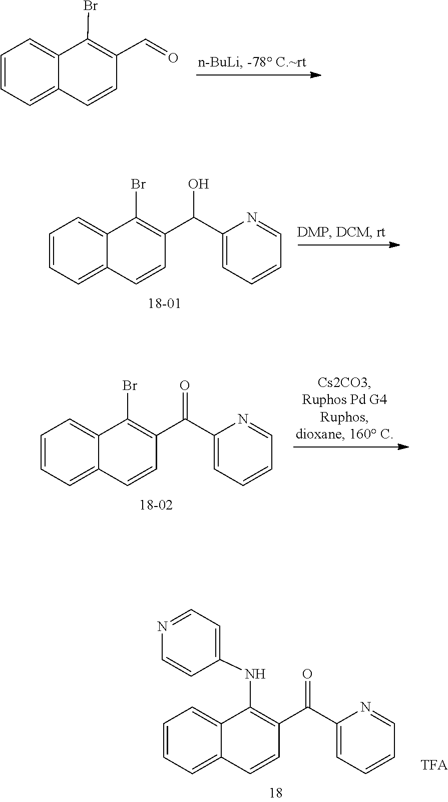 [SMILES: O=Cc1ccc2ccccc2c1Br ; OC(c1ccccn1)c1ccc2ccccc2c1Br ; O=C(c1ccccn1)c1ccc2ccccc2c1Br ; O=C(c1ccccn1)c1ccc2ccccc2c1Nc1ccncc1]

Step 1: To a solution of 2-bromopyridine (10.06 g, 64.1 mmol, 1.5 eq) in THF (150 mL) was added n-BuLi (2.5 M in hexanes, 25.6 mL, 64.1 mmol, 1.5 eq) at 0° C. The reaction mixture was stirred at −78° C. for 1 h under N₂, and then a solution of 1-bromo-2-naphthaldehyde (10 g, 42.7 mmol, 1.0 eq) was added at −78° C. The reaction mixture was stirred at room temperature for 16 h. LC-MS indicated that the reaction was complete. The reaction mixture was quenched with saturated ammonium chloride solution and extracted with DCM. The organic phase was dried over Na₂SO₄, filtered, and concentrated under reduced pressure. The crude product was purified by silica gel column chromatography to give Compound 18-01 (4.3 g) as a white solid. [M+H]⁺=314, 316. Yield: 32.2%.

Step 2: A mixture of Compound 18-01 (3.275 g, 10.4 mmol, 1.0 eq) and DMP (5.75 g, 13.55 mmol, 1.3 eq) in DCM (60 mL) was stirred at room temperature for 1 h under N₂. LC-MS indicated that the reaction was complete. The reaction mixture was poured into saturated sodium bicarbonate solution (60 mL) and extracted with DCM. The organic phase was dried over Na₂SO₄, filtered, and concentrated under reduced pressure. The crude product was purified by silica gel column chromatography to give Compound 18-02 (2.98 g) as a white solid. Yield: 92.1%.

Step 3: A mixture of Compound 18-02 (100 mg, 0.322 mmol, 1.0 eq), 4-aminopyridine (54 mg, 0.579 mmol, 1.8 eq), Cs₂CO₃ (262 mg, 0.805 mmol, 2.5 eq), RuPhos Pd G4 (27 mg, 0.0322 mmol, 0.1 eq), and RuPhos (30 mg, 0.0644 mmol, 0.2 eq) in dioxane (2 mL) was stirred at 160° C. for 1 h under N₂ under microwave irradiation. LC-MS indicated that the reaction was complete. The reaction was conducted in parallel in six batches, and the reaction mixtures were combined for joint processing. The combined reaction mixtures were concentrated and purified by silica gel column chromatography to give the crude product (82 mg). The crude product was treated with DCM (2 mL) and TFA (1 mL), and then purified by preparative HPLC to give Compound 18 (29.6 mg) as a white solid. Yield: 3.5%.

Overall yield: 1%.

### Example 13. Synthesis of Compound 19

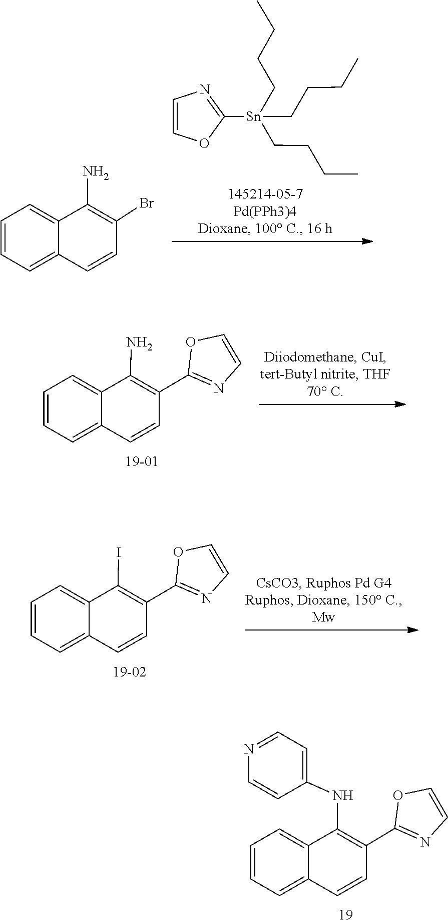 [SMILES: Nc1c(Br)ccc2ccccc12 ; CCC[CH2][Sn]([CH2]CCC)([CH2]CCC)[c]1ncco1 ; Nc1c(-c2ncco2)ccc2ccccc12 ; Ic1c(-c2ncco2)ccc2ccccc12 ; c1ccc2c(Nc3ccncc3)c(-c3ncco3)ccc2c1]

Step 1: A mixture of 1-amino-2-bromonaphthalene (0.3 g, 1.0 eq) in dioxane was treated with 2-(tributylstannyl)oxazole (0.96 g, 0.002 mol, 2.0 eq) and tetrakis(triphenylphosphine)palladium(0) (156 mg, 0.1 eq). The resulting suspension was stirred at 105° C. for 16 h under a nitrogen atmosphere. LC-MS indicated that the reaction was complete. The reaction mixture was filtered and concentrated to give the crude product, which was purified by silica gel column chromatography (PE/EA=5:1) to afford Compound 19-01 (200 mg) as a white solid. Yield: 70.6%.

Step 2: A solution of Compound 19-01 (100 mg, 0.47 mmol, 1.0 eq) in THF was treated with diiodomethane (6.4 g, 23.8 mmol, 2.0 eq), CuI (109 mg, 0.57 mmol, 1.2 eq), and tert-butyl nitrite (147 mg, 1.43 mmol, 3.0 eq) under N₂. The reaction mixture was stirred at 70° C. for 2 h. LC-MS indicated that the reaction was complete. The reaction mixture was diluted with water and extracted with ethyl acetate. The combined organic layers were washed with brine, dried over Na₂SO₄, filtered, and concentrated under reduced pressure to afford crude Compound 19-02. The crude product was purified by silica gel column chromatography (PE/EA=10:1) to afford Compound 19-02 (80 mg). Yield: 52.6%.

Step 3: A solution of Compound 19-02 (80 mg, 0.25 mmol, 1.0 eq) in 1,4-dioxane was treated with 4-aminopyridine (28 mg, 0.3 mmol, 1.2 eq), Cs₂CO₃ (203 mg, 0.62 mmol, 2.5 eq), RuPhos Pd G4 (11 mg, 0.012 mmol, 0.05 eq), and RuPhos (12 mg, 0.025 mmol, 0.1 eq) under N₂. The reaction mixture was stirred at 150° C. for 40 min under microwave irradiation. LC-MS indicated that the reaction was complete. The reaction mixture was filtered and concentrated to give the crude product, which was purified by preparative HPLC to afford Compound 19 (4 mg) as a white solid. Yield: 5.6%.

Overall yield: 2.1%.

### Example 14. Synthesis of Compound 20

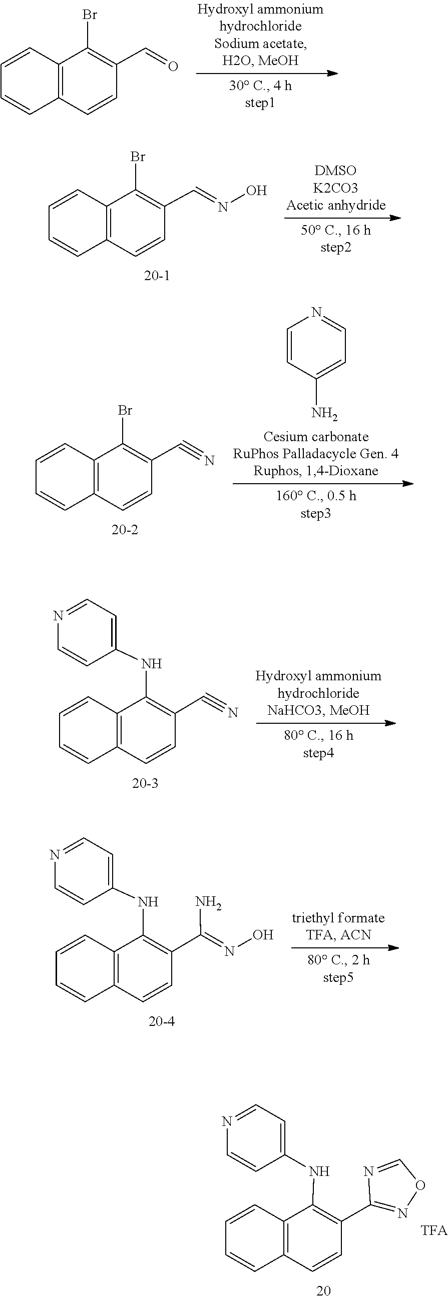

Step 1: A mixture of 1-bromo-2-naphthaldehyde (5 g, 0.021 mol, 1 eq), hydroxylamine hydrochloride (2 g, 0.03 mol, 1.4 eq), and sodium acetate (3.5 g, 0.043 mol, 2 eq) in MeOH (75 mL) and H₂O (20 mL) was stirred at 30° C. for 4 h under N₂. LC-MS indicated that the reaction was complete. The reaction mixture was diluted with H₂O and extracted with DCM (50 mL×2). The organic layers were combined, dried over Na₂SO₄, and concentrated under reduced pressure. The residue was purified by fast silica gel column chromatography to give Compound 20-1 (4.5 g). Yield: 99%.

Step 2: To a 25 mL three-neck round-bottom flask was added DMSO (45 mL), K₂CO₃ (5 g, 0.036 mol, 2 eq), and Compound 20-1 (4.5 g, 0.018 mol, 1 eq). The reaction mixture was cooled to 0° C. under a nitrogen atmosphere and acetic anhydride (3.7 g, 0.036 mol, 2 eq) was added. The reaction mixture was stirred at 50° C. for 16 h under N₂, LC-MS indicated that the reaction was complete. The reaction mixture was diluted with H₂O and extracted with EA (100 mL×2). The organic layers were combined, dried over Na₂SO₄, and concentrated under reduced pressure. The residue was purified by fast silica gel column chromatography to give Compound 20-2 (5.1 g).

Yield: 100%.

Step 3: A mixture of Compound 20-2 (1 g, 4.32 mmol, 1 eq), 4-aminopyridine (490 mg, 5.2 mmol, 1.2 eq), cesium carbonate (3.5 g, 10.8 mmol, 2.5 eq), RuPhos Palladacycle Gen. 4 (184 mg, 0.216 mmol, 0.05 eq), and dicyclohexyl-[2-[2,6-di(propan-2-yloxy)phenyl]phenyl]phosphane (201 mg, 0.432 mmol, 0.1 eq) in 1,4-dioxane (20 mL) was stirred at 160° C. for 0.5 h under microwave irradiation. The reaction was repeated five times under identical conditions. LC-MS indicated that the reaction was complete. The combined reaction mixtures were filtered and concentrated under reduced pressure. The residue was purified by column chromatography on silica gel to afford Compound 20-3 (1.1 g). Yield: 94%.

Step 4: A mixture of Compound 20-3 (500 mg, 2 mmol, 1 eq), hydroxylamine hydrochloride (210 mg, 3.05 mmol, 1.5 eq), and NaHCO₃ (256 mg, 3.05 mmol, 1.5 eq) in MeOH (20 mL) was stirred at 80° C. for 36 h. LC-MS indicated that the reaction was complete. The reaction mixture was concentrated under reduced pressure. The residue was successively treated with DCM (10 mL), MeOH (20 mL), and H₂O (15 mL), each followed by stirring for 0.5 h, filtration, and concentration under reduced pressure to afford Compound 20-4 (97 mg). Yield: 16.9%.

Step 5: A mixture of Compound 20-4 (110 mg, 0.39 mmol, 1 eq), triethyl orthoformate (55 mg, 0.51 mmol, 1.3 eq), and TFA (2 mg, 0.02 mmol, 0.05 eq) in ACN (10 mL) was stirred at 80° C. for 2 h. LC-MS indicated that the reaction was complete. The reaction mixture was concentrated under reduced pressure. The residue was treated with DCM (2 mL) and TFA (1 mL) and purified by preparative HPLC to afford Compound 2 (36 mg). Yield: 32%.

Overall yield: 5.04%.

### Example 15. Synthesis of Compound 21

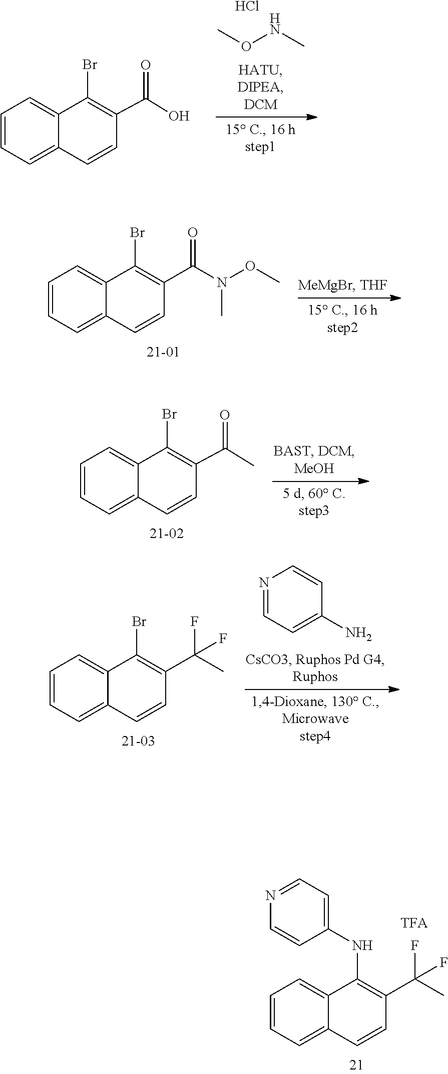

Step 1: To a solution of 1-bromo-2-naphthoic acid (2.5 g, 9.96 mmol, 1 eq) in DCM (25 mL) was added N-methoxy-methanamine hydrochloride (1.94 g, 19.91 mmol, 2 eq), DIPEA (5.15 g, 39.84 mmol, 4 eq), and HATU (4.54 g, 11.95 mmol, 1.2 eq) under N₂. The reaction mixture was stirred at 25° C. for 16 h. LC-MS indicated that the reaction was complete. The reaction mixture was purified by fast silica gel column chromatography to afford Compound 21-01 (2.97 g). Yield: 100%.

Step 2: To a solution of Compound 21-01 (2.97 g, 10.14 mmol, 1 eq) in THF (30 mL), the suspension was cooled to −78° C. and a 3.0 M solution of methylmagnesium bromide in THF (3.5 mL) was added. The reaction mixture was stirred at 25° C. for 16 h. LC-MS indicated that the reaction was complete. A saturated solution of NH₄Cl was added and the mixture was extracted with EA. The combined organic layers were dried over Na₂SO₄ and concentrated in vacuo. The crude material was purified by chromatography on silica gel to afford Compound 21-02 (1.2 g) as a white solid. Yield: 48%.

Step 3: To a solution of Compound 21-02 (200 mg, 0.81 mmol, 1 eq) in DCM (2 mL) was added BAST (1.434 g, 6.48 mmol, 8 eq) and one drop of MeOH under N₂. The mixture was stirred at 60° C. for 5 d. GC-MS indicated that the reaction was complete. The mixture was cooled to room temperature and poured into ice water, then neutralized with saturated NaHCO₃ solution (pH=7-8). The mixture was extracted with EA. The combined organic layers were washed with brine, dried over anhydrous Na₂SO₄, and concentrated to give a crude residue. The crude material was purified by chromatography on silica gel to afford Compound 21-03 (76 mg) as a white solid.

Yield: 27.46%.

Step 4: To a solution of Compound 21-03 (56 mg, 0.207 mmol, 1 eq) in 1,4-dioxane (6 mL) was added 4-aminopyridine (23.4 mg, 0.2484 mmol, 1.2 eq), Cs₂CO₃ (169 mg, 0.519 mmol, 2.5 eq), RuPhos Pd G4 (21 mg, 0.02484 mmol, 0.12 eq), and RuPhos (97 mg, 0.208 mmol, 1 eq) under N₂. The mixture was stirred for 20 min at 130° C. under microwave irradiation. LC-MS indicated that some starting material remained. The reaction mixture was filtered and concentrated to give the crude product. The crude was treated with DCM (1 mL) and TFA (0.5 mL) and stirred for 2 min at 25° C. The reaction mixture was concentrated under vacuum and the residue was purified by preparative HPLC to give Compound 21 (8.7 mg) as a white solid. Yield: 10.61%.

Overall yield: 1.40%.

### Example 16. Synthesis of Compound 22

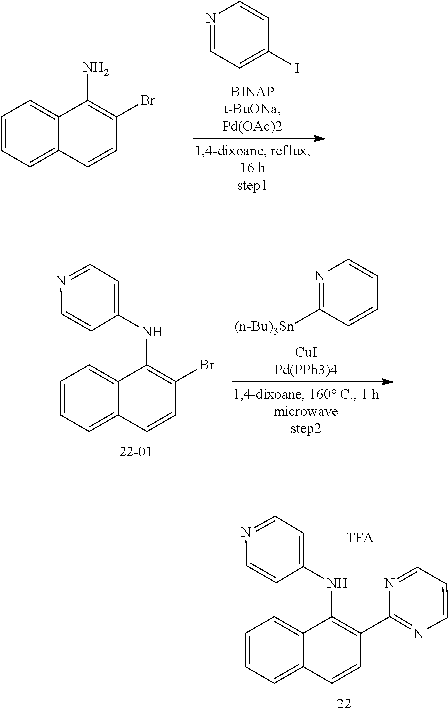

Step 1: To a 250 mL three-neck round-bottom flask was added 1,4-dioxane (80 mL), 1-amino-2-bromonaphthalene (4 g, 18 mmol, 1 eq), t-BuONa (6.05 g, 63 mmol, 3.5 eq), Pd(OAc)₂ (0.403 g, 1.8 mmol, 0.1 eq), 4-iodopyridine (3.7 g, 18 mmol, 1 eq), and BINAP (1.12 g, 1.8 mmol, 0.1 eq). The reaction mixture was stirred at reflux for 16 h. LC-MS indicated that the reaction was complete. The mixture was extracted with EA. The organic phase was dried over Na₂SO₄, concentrated, and purified by fast silica gel column chromatography to give Compound 22-01 (4 g) as a brown solid. Yield: 74.32%. [M+H]⁺=299.0/301.0.

¹H NMR (400 MHz, DMSO-d₆) δ 8.86 (s, 1H), 8.15-7.99 (m, 3H), 7.94-7.78 (m, 3H), 7.67-7.50 (m, 2H), 6.37 (s, 2H).

Step 2: To a 10 mL vial was added 1,4-dioxane (5 mL), Compound 22-01 (100 mg, 0.335 mmol, 1 eq), CuI (7 mg, 0.034 mmol, 0.1 eq), 2-(tributylstannyl)pyrimidine (137 mg, 0.369 mmol, 1.1 eq), and Pd(PPh₃)₄ (40 mg, 0.034 mmol, 0.1 eq). The reaction mixture was purged with nitrogen and stirred for 1 h at 160° C. under microwave irradiation. LC-MS indicated that the reaction was complete. The reaction mixture was filtered through a pad of Celite, and TFA (0.5 mL) was added at room temperature. The filtrate was concentrated to dryness and purified by preparative TLC followed by preparative HPLC to give Compound 22 (100 mg) as a light yellow solid. Yield: 72.45%.

Overall yield: 53.85%.

### Example 17. Synthesis of Compound 23

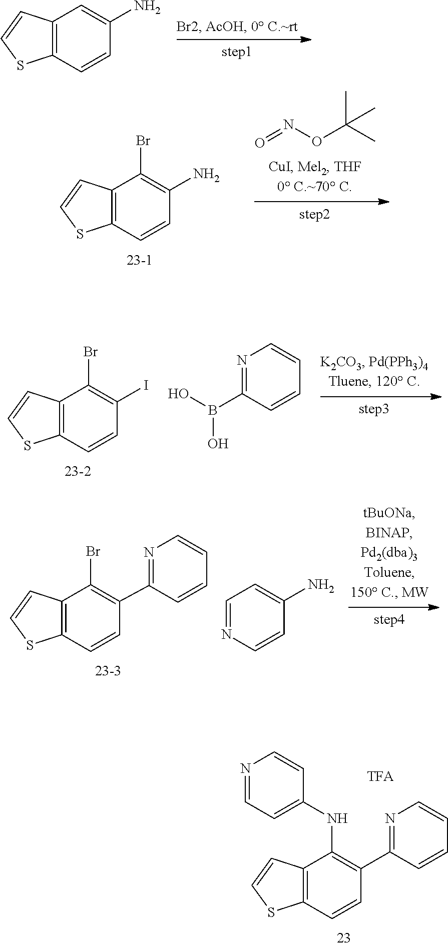

Step 1: To a solution of benzo[b]thiophen-5-amine (2 g, 13.4 mmol, 1 eq) in AcOH (30 mL) was added Br₂ (2.36 g, 14.74 mmol, 1.1 eq) at 0° C. under N₂. The reaction mixture was stirred at 20° C. for 2 h. TLC indicated that the reaction was complete. The reaction mixture was poured into water (50 mL) and extracted with EA (20 mL×3). The organic layers were combined, concentrated under reduced pressure, and purified by column chromatography to afford Compound 23-1 (1.6 g). Yield: 65.3%.

[M+H]⁺=228.0, 230.0.

Step 2: To a solution of Compound 23-1 (1.6 g, 0.44 mmol, 1.0 eq), CuI (1.6 g, 0.53 mmol, 1.2 eq), and Mel (3.76 g, 0.88 mmol, 2.0 eq) in THF (30 mL) was added tert-butyl nitrite (1.08 g, 0.66 mmol, 1.5 eq) at 0° C. under N₂. The reaction mixture was stirred at 70° C. for 2 h. TLC indicated that the reaction was complete. The reaction mixture was poured into water (50 mL) and filtered. The filtrate was extracted with EA (10 mL×3). The organic layers were combined, concentrated under reduced pressure, and purified by column chromatography to afford Compound 23-2 (500 mg). Yield: 21%.

GC-MS: 337.8, 339.8.

Step 3: To a solution of Compound 23-2 (500 mg, 1.47 mmol, 1.0 eq) in toluene (10 mL) was added pyridin-2-ylboronic acid (453.25 mg, 3.69 mmol, 2.5 eq), K₂CO₃ (815 mg, 5.9 mmol, 4.0 eq), and Pd(PPh₃)₄ (170 mg, 0.15 mmol, 0.1 eq). The reaction mixture was stirred at 120° C. for 12 h. LC-MS indicated that the reaction was complete. The reaction mixture was filtered and concentrated under reduced pressure. The crude product was purified by column chromatography to afford Compound 23-3 (110 mg). Yield: 25.7%. [M+H]⁺=290.0, 292.0.

Step 4: To a solution of Compound 23-3 (100 mg, 0.34 mmol, 1 eq) in toluene (2 mL) was added pyridin-4-amine (64.87 mg, 0.69 mmol, 2.0 eq), t-BuONa (99.36 mg, 1.03 mmol, 3.0 eq), Pd₂(dba)₃ (31.56 mg, 0.03 mmol, 0.1 eq), and BINAP (42.92 mg, 0.07 mmol, 0.2 eq). The reaction mixture was stirred at 150° C. for 50 min under microwave irradiation. LC-MS indicated that the reaction was complete. The reaction mixture was filtered and concentrated under reduced pressure, and the mixture was adjusted to pH=3 with TFA. The crude product was purified by preparative HPLC to give Compound 23 (7 mg) as a white solid. Yield: 5.1%.

Overall yield: 0.18%.

### Example 18. Synthesis of Compound 24

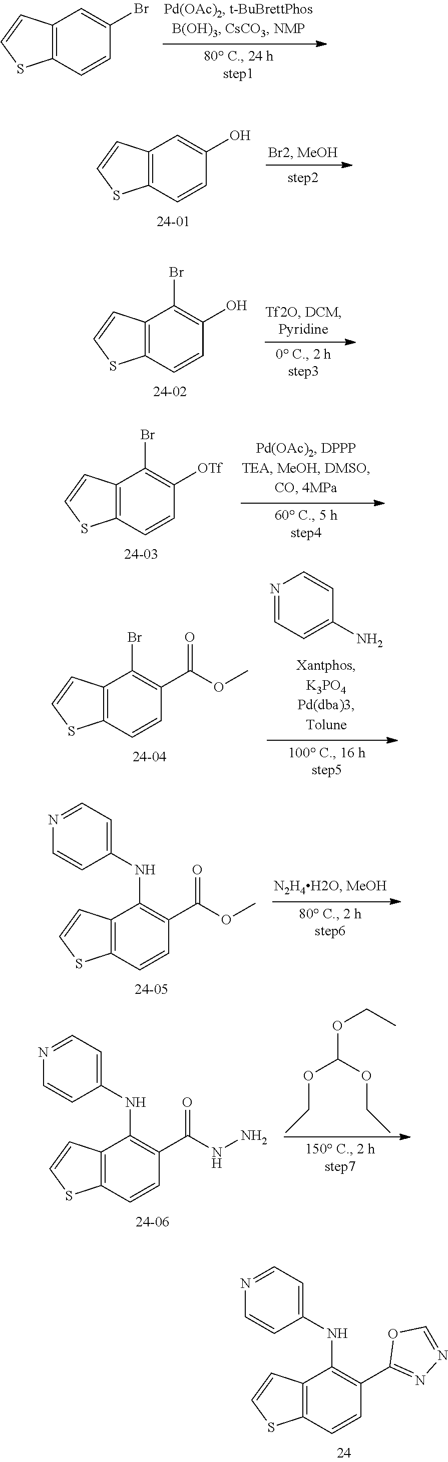

Step 1: A mixture of 5-bromobenzothiophene (18 g, 84.5 mmol, 1.0 eq), B(OH)₃ (7.83 g, 127 mmol, 1.5 eq), Cs₂CO₃ (55.06 g, 169 mmol, 2.0 eq), t-BuBrettPhos (5.12 g, 10.6 mmol, 0.125 eq), and Pd(OAc)₂ (948.6 mg, 4.23 mmol, 0.05 eq) in NMP (180 mL) was stirred at 82° C. for 48 h under N₂. GC-MS indicated that the reaction was complete. The mixture was diluted with water and extracted with EA. The organic layers were combined, dried over Na₂SO₄, and concentrated to dryness to give the crude product. The crude material was purified by silica gel column chromatography to afford Compound 24-01 (5 g) as a light yellow solid. Yield: 39.37%. [M+H]⁺=151.1.

Step 2: Compound 24-01 (5 g, 33.3 mmol, 1.0 eq) was dissolved in MeOH (100 mL), and at −78° C. a solution of Br₂ (5.33 g, 33.3 mmol, 1.0 eq) in MeOH (50 mL) was added dropwise. The reaction mixture was stirred at −78° C. for 2 h. TLC indicated that the reaction was complete. Saturated NaHCO₃ solution (100 mL) was added and the mixture was extracted with EA. The organic layers were combined, dried over Na₂SO₄, and concentrated to give the crude product, which was purified by silica gel column chromatography to afford Compound 24-02 (4 g) as a white solid. Yield: 52.63%.

[M+H]⁺=228.9.

¹H NMR (400 MHz, CDCl₃) δ 7.68 (d, J=8.6 Hz, 1H), 7.53 (d, J=5.5 Hz, 1H), 7.34 (d, J=5.5 Hz, 1H), 7.09 (d, J=8.6 Hz, 1H), 5.57 (s, 1H).

Step 3: A mixture of Compound 24-02 (1.5 g, 6.58 mmol, 1.0 eq), pyridine (780.72 mg, 9.87 mmol, 1.5 eq), and trifluoromethanesulfonic anhydride (2.04 g, 7.24 mmol, 1.1 eq) in DCM (15 mL) was stirred at 0° C. for 2 h under N₂. TLC indicated that the reaction was complete. The mixture was diluted with water and extracted with EA. The organic layers were combined, dried over Na₂SO₄, and concentrated to give the crude product, which was purified by silica gel column chromatography to afford Compound 24-03 (1.8 g) as a white solid. Yield: 75%. [M−H]=358.9.

Step 4: A mixture of Compound 24-03 (1 g, 2.78 mmol, 1.0 eq), DPPP (275 mg, 0.67 mmol, 0.24 eq), TEA (534.5 mg, 5.28 mmol, 1.9 eq), and Pd(OAc)₂ (125 mg, 0.556 mmol, 0.2 eq) in MeOH (25 mL) and DMSO (25 mL) was stirred at 60° C. for 6 h under CO (4 MPa) in an autoclave. The mixture was diluted with water and extracted with EA. The organic layers were combined, dried over Na₂SO₄, and concentrated to give the crude product. The crude material was purified by silica gel column chromatography (PE:EA=97:3) to afford Compound 24-04 (441 mg) as a white solid. Yield: 58.80%.

[M+H]⁺=270.9.

¹H NMR (400 MHz, CDCl₃) δ 7.83 (dd, J=8.4, 0.6 Hz, 1H), 7.75 (d, J=8.4 Hz, 1H), 7.66 (dd, J=5.6, 0.6 Hz, 1H), 7.57 (d, J=5.6 Hz, 1H), 3.97 (s, 3H).

Step 5: A mixture of Compound 24-04 (200 mg, 0.741 mmol, 1.0 eq), 4-aminopyridine (84 mg, 0.889 mmol, 1.2 eq), K₃PO₄ (220.2 mg, 1.04 mmol, 1.4 eq), Xantphos (58 mg, 0.1 mmol, 0.14 eq), and Pd₂(dba)₃ (30 mg, 0.033 mmol, 0.05 eq) in toluene (10 mL) was stirred at 100° C. for 16 h under N₂. The reaction mixture was diluted with EA, filtered through a pad of Celite, and concentrated under reduced pressure. The crude product was purified by silica gel column chromatography (DCM:MeOH=20:1) to afford Compound 24-05 (158 mg) as a white solid.

Yield: 70.06%.

[M+H]⁺=285.1.

¹H NMR (400 MHz, CDCl₃) δ 9.34 (s, 1H), 8.31 (d, J=6.2 Hz, 2H), 7.98 (d, J=8.6 Hz, 1H), 7.66 (d, J=8.6 Hz, 1H), 7.39 (d, J=5.6 Hz, 1H), 7.07 (d, J=5.6 Hz, 1H), 6.69 (d, J=6.3 Hz, 2H), 3.94 (s, 3H).

Step 6: A mixture of Compound 24-05 (158 mg, 0.556 mmol, 1.0 eq) and hydrazine hydrate solution (1.74 mL) in MeOH (3.5 mL) was stirred at 80° C. for 2 h under N₂. TLC indicated that the reaction was complete. The reaction mixture was filtered through a pad of Celite. The filter cake was air-dried to provide Compound 24-06 (125 mg), which was used in the next step without further purification. Yield: 79.11%. [M+H]⁺=285.1.

Step 7: A mixture of Compound 24-06 (65 mg, 0.228 mmol, 1.0 eq) and triethyl orthoformate (0.65 mL) was stirred at 150° C. for 2 h under N₂. LC-MS indicated that the reaction was complete. The crude product was purified by preparative HPLC to afford Compound 24 (11 mg) as a light yellow solid. Yield: 16.30%.

Overall yield: 0.83%.

Characterization data of some compounds are listed in Table 2 below.

TABLE 2

| Compound No. | Characterization data |
|---|---|
| 1 | LC-MS: [M—C2HF3O2 + 1]+ = 263.2 |
| | 1H NMR (400 MHz, DMSO) δ 13.61 (s, 1H), 10.73-10.60 |
| | (m, 1H), 8.44-8.10 (m, 4H), 7.97-7.88 (m, 2H), 7.77-7.64 |
| | (m, 2H), 6.72 (d, J = 426.3 Hz, 2H), 2.58-2.56 (m, 3H). |
| 2 | LCMS: [M—C2HF3O2 + 1]⁺ = 314.1 |
| | ¹H NMR (400 MHz, DMSO) δ 13.44 (s, 1H), 10.17 (s, 1H), |
| | 9.71 (s, 1H), 8.35 (s, 1H), 8.08 (d, J = 9.0 Hz, 3H), 7.82 (s, 1H), |
| | 7.66 (d, J = 6.9 Hz, 1H), 7.57 (s, 2H), 7.33 (s, 1H), 6.04 (s, 1H), |
| | 3.09 (s, 3H). |
| 3 | LC-MS: [M—C2HF3O2 + 1]+ = 264.1 |
| | 1H NMR (400 MHz, DMSO) δ 13.65 (s, 1H), 10.68 (s, 1H), |
| | 9.53 (s, 1H), 8.40 (dd, J = 6.3, 2.8 Hz, 1H), 8.25 (s, 2H), 8.02- |
| | 7.87 (m, 3H), 6.85 (s, 2H), 2.71 (s, 3H). |
| 4 | LC-MS: [M—C2HF3O2 + 1]+ = 297.1/299.1. |
| | 1H NMR (400 MHz, DMSO) δ 8.23 (dd, J = 30.9, 23.0 Hz, |
| | 4H), 7.94 (dd, J = 11.4, 5.2 Hz, 2H), 7.76 (dd, J = 8.8, 2.0 Hz, |
| | 1H), 7.31 (t, J = 46.2 Hz, 1H), 6.20 (s, 1H), 2.57 (s, 3H). |
| 5 | LC-MS: [M—C2HF3O2 + 1]+ = 265.1 |
| | 1H NMR (400 MHz, DMSO) δ 13.27 (s, 2H), 10.65 (s, 1H), 8.2 |
| | 1 (d, J = 5.9 Hz, 2H), 8.14 (d, J = 8.5 Hz, 2H), 8.01 (d, J = 8.6 |
| | Hz, 1H), 7.90 (d, J = 8.4 Hz, 1H), 7.74 (t, J = 7.2 Hz, 1H), 7.67 |
| | (t, J = 7.3 Hz, 1H), 6.69 (s, 2H). |
| 6 | LC-MS: [M—C2HF3O2 + 1]+ = 299.1 |
| | 1H NMR (400 MHz, DMSO) δ 13.72 (s, 1H), 10.75 (s, 1H), |
| | 8.57-8.30 (m, 2H), 8.24 (d, J = 8.2 Hz, 1H), 8.14 (d, J = 8.8 |
| | Hz, 2H), 7.91 (d, J = 8.5 Hz, 1H), 7.83 (t, J = 7.5 Hz, 1H), 7.78- |
| | 7.68 (m, 1H), 7.46 (s, 1H), 6.04 (s, 1H), 3.34 (s, 3H). |
| 7 | LC-MS: [M—C2HF3O2 + 1]+ = 289.1 |
| | 1H NMR (400 MHz, DMSO) δ 13.59 (s, 1H), 10.82 (s, 1H), |
| | 9.43 (s, 1H), 8.49-8.26 (m, 2H), 8.24-8.17 (m, 2H), 8.16- |
| | 8.02 (m, 1H), 7.96 (d, J = 8.4 Hz, 1H), 7.79 (t, J = 7.4 Hz, 1H), |
| | 7.73 (t, J = 7.6 Hz, 1H), 7.40 (s, 1H), 6.14 (s, 1H). |
| 8 | LC-MS: [M—C2HF3O2 + 1]+ = 269.1 |
| | 1H NMR (400 MHz, DMSO) δ 13.57 (s, 1H), 10.69 (s, 1H), |
| | 8.35-8.20 (m, 3H), 7.94 (dd, J = 19.3, 7.0 Hz, 2H), 7.27 (d, |
| | J = 5.6 Hz, 1H), 6.78 (s, 2H), 2.57 (s, 3H). |
| 9 | LC-MS: [M—C2HF3O2 + 1]+ = 269.1 |
| | 1H NMR (400 MHz, DMSO) δ 13.66 (s, 1H), 10.77 (s, 1H), |
| | 8.28 (d, J = 7.1 Hz, 2H), 8.10 (d, J = 8.3 Hz, 1H), 8.02 (dd, J = |
| | 15.0, 6.9 Hz, 2H), 7.67 (d, J = 5.4 Hz, 1H), 6.80 (s, 2H), 2.58 |
| | (s, 3H). |
| 10 | LC-MS: [M—C2HF3O2 + 1]+ = 251.1. |
| | 1H NMR (400 MHz, DMSO) δ 13.45 (s, 1H), 10.31 (s, 1H), |
| | 8.31 (s, 1H), 8.09 (d, J = 9.1 Hz, 2H), 8.00 (d, J = 8.1 Hz, 1H), |
| | 7.73 (d, J = 8.4 Hz, 1H), 7.66 (d, J = 9.2 Hz, 1H), 7.56 (t, J = |
| | 7.2 Hz, 1H), 7.46 (t, J = 7.5 Hz, 1H), 7.32 (s, 1H), 6.16 (s, 1H), |
| | 3.93 (s, 3H). |
| 11 | LCMS: [M—C2HF3O2 + 1]+ = 317.1 |
| | 1H NMR (400 MHz, DMSO + D2O) δ 8.24 (d, J = 6.9 Hz, 1H), |
| | 8.11 (t, J = 8.3 Hz, 2H), 7.96 (t, J = 6.9 Hz, 2H), 7.75-7.66 |
| | (m, 2H), 7.62 (d, J = 7.2 Hz, 1H), 7.39 (d, J = 5.1 Hz, 1H), 5.84 |
| | (d, J = 5.3 Hz, 1H). |
| 12 | LCMS: [M—C2HF3O2 + 1]+ = 246.1 |
| | 1H NMR (400 MHz, DMSO) δ 13.87 (s, 1H), 11.06 (s, 1H), |
| | 8.36 (d, J = 6.9 Hz, 2H), 8.23 (t, J = 9.4 Hz, 2H), 7.98 (t, J = 8.9 |
| | Hz, 2H), 7.84 (t, J = 7.5 Hz, 1H), 7.76 (t, J = 7.6 Hz, 1H), 7.17- |
| | 6.65 (m, 2H). |
| 13 | LC-MS: [M—C2HF3O2 + 1]+ = 325.1 |
| | 1H NMR (400 MHz, DMSO) δ 13.48 (s, 1H), 10.54 (s, 1H), |
| | 8.19 (dd, J = 21.4, 15.0 Hz, 4H), 7.84 (d, J = 8.4 Hz, 1H), 7.78- |
| | 7.60 (m, 6H), 7.46 (t, J = 7.7 Hz, 2H), 7.00 (s, 1H), 6.28 (s, |
| | 1H). |
| 14 | LC-MS: [M—C2HF3O2 + 1]+ = 305.0 |
| | 1H NMR (400 MHz, DMSO) δ 13.72 (s, 1H), 10.68 (s, 1H), |
| | 8.38 (t, J = 29.4 Hz, 3H), 8.05 (dd, J = 14.0, 7.1 Hz, 2H), 7.29 |
| | (dd, J = 5.6, 0.5 Hz, 3H), 3.28 (s, 3H). |
| 17 | LC-MS: [M + H]⁺ = 298.1 |
| | ¹H NMR (400 MHz, DMSO) δ 13.40 (s, 1H), 10.68 (s, 1H), |
| | 8.65 (d, J = 4.2 Hz, 1H), 8.38-8.08 (m, 3H), 8.02-7.77 (m, |
| | 4H), 7.74-7.60 (m, 3H), 7.36 (dd, J = 7.1, 5.2 Hz, 1H), 7.20 |
| | (d, J = 4.9 Hz, 1H), 6.06 (d, J = 5.2 Hz, 1H). |
| 18 | LC-MS: [M + H]+ = 326.2. |
| | ¹H NMR (400 MHz, DMSO) δ 13.63 (s, 1H), 10.49 (s, 1H), |
| | 8.44 (d, J = 4.5 Hz, 1H), 8.18 (d, J = 8.4 Hz, 4H), 7.98 (dt, J = |
| | 11.0, 7.6 Hz, 2H), 7.81 (d, J = 8.4 Hz, 1H), 7.74 (t, J = 7.4 Hz, |
| | 2H), 7.67 (t, J = 7.0 Hz, 1H), 7.56 (dd, J = 8.5, 3.2 Hz, 1H), |
| | 6.98-6.79 (m, 1H), 6.23 (s, 1H). |
| 19 | LC-MS: [M + H]+ = 288.1 |
| | 1H NMR (400 MHz, DMSO) δ 13.63 (s, 1H), 10.77 (s, 1H), |
| | 8.44-7.86 (m, 7H), 7.71 (ddd, J = 15.3, 13.8, 6.8 Hz, 2H), |
| | 7.57-7.09 (m, 2H), 6.13 (s, 1H). |
| 20 | [M + H]+ = 289.1 |
| | 1H NMR (400 MHz, DMSO) δ 13.66 (s, 1H), 10.79 (s, 1H), |
| | 9.77 (s, 1H), 8.53-7.92 (m, 6H), 7.81-7.65 (m, 2H), 7.36 (s, |
| | 1H), 6.09 (s, 1H). |
| 21 | LC-MS: [M + H]+ = 285.1 |
| | ¹H NMR (400 MHz, DMSO-d6) δ 13.59 (s, 1H), 10.62 (s, 1H), |
| | 8.38 (d, J = 7.0 Hz, 1H), 8.22 (d, J = 8.7 Hz, 1H), 8.16 (d, J = |
| | 8.0 Hz, 1H), 8.07 (d, J = 7.0 Hz, 1H), 7.81 (dd, J = 14.1, 8.5 |
| | Hz, 2H), 7.74-7.61 (m, 2H), 7.42 (dd, J = 7.0, 2.6 Hz, 1H), |
| | 5.95 (dd, J = 7.0, 2.5 Hz, 1H), 2.03 (t, J = 19.1 Hz, 3H). |
| 22 | LC-MS: [M + H]⁺ = 299.2 |
| | ¹H NMR (400 MHz, DMSO) δ 13.45 (s, 1H), 10.71 (s, 1H), |
| | 8.89 (d, J = 4.9 Hz, 2H), 8.27 (s, 1H), 8.22-8.11 (m, 3H), 7.99 |
| | (s, 1H), 7.92 (d, J = 8.2 Hz, 1H), 7.69 (dtd, J = 16.5, 6.9, 1.2 |
| | Hz, 2H), 7.46 (t, J = 4.9 Hz, 1H), 7.25 (s, 1H), 6.05 (s, 1H). |
| 23 | LC-MS: [M + H]+ = 304.1 |
| | 1H NMR (400 MHz, DMSO) δ 8.66 (d, J = 4.3 Hz, 1H), 8.25 |
| | (d, J = 8.4 Hz, 1H), 8.10 (d, J = 66.1 Hz, 2H), 7.92 (dd, J = |
| | 10.3, 3.6 Hz, 2H), 7.75 (d, J = 8.4 Hz, 1H), 7.67 (d, J = 7.9 Hz, |
| | 1H), 7.44 (dd, J = 7.0, 5.5 Hz, 1H), |
| 24 | LC-MS: [M + H]+ = 295.1 |
| | ¹H NMR (400 MHz, DMSO-d6) δ 9.30 (s, 1H), 9.06 (s, 1H), |
| | 8.12 (d, J = 7.0 Hz, 3H), 7.94 (d, J = 8.5 Hz, 1H), 7.89 (d, J = |
| | 5.6 Hz, 1H), 7.23 (d, J = 5.5 Hz, 1H), 6.54 (dd, J = 4.9, 1.4 Hz, |
| | 2H). |

### Biological Assays

### Example 1. α2A and α2C AR Agonist EC50 FLIPR Assay

This experimental protocol involved cell seeding and a FLIPR assay using the α2A or α2C AR cell line hosted in HEK293 cells. The growth media used is DMEM (11965-092, Gibco) supplemented with 10% FBS (FSP500, Excell), 300 g/mL G418 (10131-027, Gibco), and 2 g/mL Blasticidin S HCl (BS) (A11139-03, Gibco).

On Day 1, the cell seeding process started with the removal of the culture medium, followed by rinsing the cells with DPBS (21-031-CVC, Corning). Cells were then treated with 0.05% EDTA-Trypsin (25300-062, Gibco), incubated at 37° C. for 1-2 minutes, and monitored under an inverted microscope. The cells were detached, resuspended in growth media, and centrifuged at room temperature at 1000 rpm for 5 minutes. After discarding the supernatant, the cell pellet was resuspended in growth media to a concentration of 10×105 cells per mL. This suspension was added to 384-well plates (19-Jul-38, Greiner) at 20 mL per well and incubated overnight at 37° C. in 5% CO₂.

On Day 2, the FLIPR assay began with the preparation of the assay buffer comprising 20 mM HEPES (15630-106, Invitrogen), 1×HBSS (14025-076, Invitrogen), and 0.5% BSA (B2064, Sigma). A 250 mM Probenecid solution was prepared in this buffer. The Fluo-4 Direct™ Loading Buffer was made by dissolving Fluo-4 Direct™ crystals (F10471, Invitrogen) in the FLIPR Assay Buffer and adding Probenecid. The buffer was then vortexed and allowed to stand for over 5 minutes, shielded from light. For the FLIPR procedure, testing compounds for agonist tests were serially diluted and transferred to a 384-well compound plate (25-Jan-39, Greiner). The cell plate was then treated with 2×Fluo-4 Direct™ loading buffer and incubated for 50 minutes at 37° C. in a 5% CO₂ atmosphere, followed by 10 minutes at room temperature. Subsequently, the FLIPR assay buffer was added to the compound plate, which is then centrifuged.

The cell plate was analyzed in the FLIPR Tetra+ System for fluorescence signals. For the agonist test, reference compounds were added to the cell plates, and fluorescence was measured. The “Max-Min” calculation began from Read 1 to the maximum allowed. The data were analyzed using Prism software to calculate activation percentage for agonists. The results were then fitted using specific models to determine EC50 for agonists.

The experimental protocol utilized various reagents and apparatus, including Penicillin/Streptomycin (100×) (SV30010, Hyclone), Poly-L-lysine hydrobromide (P1399, Sigma), and different types of 384-well plates such as the 384-Well PP 2.0 Microplate (PP-0200, LABCYTE) and 384 well Low Dead Volume Microplate (LP-0200, LABCYTE). The use of specific reference compounds like UK14304 was also integral to the assay.

### Example 2 α2A AR Binding Assay

The α2A AR Binding Assay was conducted using a stable HEK293 cell line, specifically constructed by WuXi AppTec for targeting α2A AR. This assay primarily focused on the binding activity of the radioligand [3H]-RX 821002 (PerkinElmer, NET1153250UC) to α2A AR, with the membrane concentration set at 0.5 μg/well and the radioligand concentration at 0.5 nM. Essential equipment for this assay includes Unifilter-96 GF/C filter plates (Perkin Elmer, 6005174), 96 well conical polypropylene plates (Agilent, 5042-1385), TopSeal-A sealing film (Perkin Elmer, 6050185), a MicroBeta2 reader (CNLL0153, Perkin Elemer, 1310887), and a cell harvester (UNIFILTER-96, Perkin Elemer, 1951369), all procured from Perkin Elmer. Both the assay and wash buffers consist of 50 mM Tris-HCl at a pH of 7.4 (Tris base, Sigma, T1503-1KG).

The procedure initiated with the preparation of test compounds and a reference compound, yohimbine (Sigma, Y3125), through an 8-point 4-fold serial dilution, transferring 1 L of each to the assay plate. The assay involved adding 100 μL of membrane stocks (0.5 μg/well) and 100 μL 0.5 nM of [3H]-RX 821002 to each well. After sealing, the plates were agitated at room temperature for one hour. Subsequently, the Unifilter-96 GF/C filter plates were pre-soaked with 0.3% PEI (Sigma, P3143) for at least half an hour. The reaction mixtures were then filtered and washed four times with cold wash buffer using a Perkin Elmer Cell harvester. Post-filtration, the plates were dried at 50° C. for one hour. The next step involved sealing the bottom of the filter plate wells with Perkin Elmer Unifilter-96 backing seal tape and adding 50 μL of MicroScint-O cocktail (PerkinElmer, 6013611) to each well. The top of the plates was then sealed with TopSeal-A sealing film. The trapped 3H was quantified using a Perkin Elmer MicroBeta2 Reader. The inhibition rate was calculated using the formula: % Inhibition=(1−(Assay well Average_LC)/(Average_HC-Average_LC))*100%. Finally, the data were analyzed with Prism 5.0 software, employing the “log (inhibitor) vs. response—Variable slope” model for data fitting. This comprehensive process ensured precise assessment of the binding affinity of compounds to the α2A AR.

The results of the α2A and α2C AR agonist EC50 FLIPR assay and α2A AR affinity are listed in Table 3 below.

TABLE 3

| | α2A AR agonist | α2A AR | α2C AR agonist |
|---|---|---|---|
| Compound | activity (EC50, | affinity | activity |
| No. | nM)/(IA) | (Ki, nM) | (EC50, nM) |
| 1 | >10000 | 31.19 | 75.40 |
| 2 | >10000 | | 56.32 |
| 3 | | | >10000 |
| 4 | >10000 | | 25.87 |
| 5 | | | >10000 |
| 6 | 380.95 (43.00%) | 85.12 | 30.65 |
| 7 | 136.95 (27.75%) | 13.45 | 6.88 |
| 8 | >10000 | 17.88 | 105.49 |
| 9 | >10000 | 29.19 | 83.16 |
| 10 | >10000 | | 16.69 |
| 11 | 400.08 (29.39%) | | 24.92 |
| 12 | >10000 | | 6.22 |
| 13 | >10000 | | 6.03 |
| 14 | | | 11.12 |
| 17 | >10000 | | |
| 18 | 152.09 (18.65%) | | 17.53 |
| 19 | >10000 | | 24.52 |
| 20 | | | 498.18 |
| 21 | 183.73 (51.81%) | | 19.84 |
| 22 | | | 96.39 |
| 23 | | | 58.41 |
| 24 | | | 64.41 |

IA = maximal response to the test agonist/maximal response to full agonist.

### Example 3. Inhibition of LPS-Induced Cytokine Production in Mice

The pharmacological activity of compounds of the present disclosure was evaluated in a lipopolysaccharide (LPS)-induced cytokine production model in mice.

Female C57BL/6 mice having a body weight of approximately 20±2 g were randomly assigned to experimental groups including a normal control group, a vehicle control group, and treatment groups receiving test compounds.

Test compounds (e.g., 7, 12, 13, or 14) were administered orally at a dose of 20 mg/kg. Approximately 30 minutes following administration of the test compounds, mice were challenged with LPS via intraperitoneal injection to induce systemic inflammatory responses.

At approximately 1 hour following LPS administration, blood samples were collected from the submandibular vein into anticoagulant-containing tubes. Plasma was separated by centrifugation and plasma cytokine levels were determined using a suitable immunoassay method.

Following LPS challenge, plasma cytokine levels in the vehicle-treated model group were markedly elevated compared with those observed in the normal control group, confirming successful induction of systemic inflammation. In particular, plasma TNF-α levels were substantially increased following LPS stimulation.

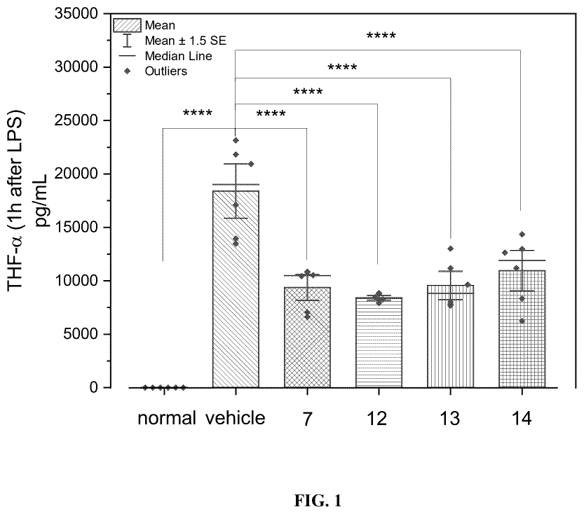

As demonstrated in FIG. 1, administration of the test compounds significantly reduced plasma TNF-α levels relative to the vehicle control group. Compounds 7, 12, 13, and 14 each demonstrated inhibitory effects on LPS-induced cytokine production.

These results demonstrate that the compounds of the present disclosure are capable of suppressing LPS-induced pro-inflammatory cytokine production in vivo. Accordingly, the compounds described herein may be useful for the treatment of diseases or conditions associated with excessive cytokine production, including sepsis, systemic inflammatory response syndrome, autoimmune diseases, and other inflammatory disorders.

1. A compound of formula (I): 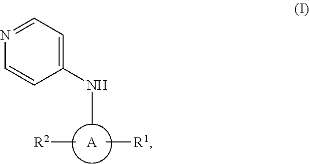
or a stereoisomer, tautomer, pharmaceutically acceptable salt or a solvate thereof,
wherein,
ring A is a bicyclic ring;
R¹ is chosen from —C₀₋₁₂ haloalkyl, —C(O)—C₀₋₁₂ alkyl, —C(O)—C₀₋₁₂ haloalkyl, —C(O)-aryl, —C(O)-heteroaryl, C₀₋₁₂ alkylene-COOH, —C(O)—NR³R^{3′}, —CN, —NR³R^{3′}, —N³C(O) R^{3′}, —NR³—SO₂—R^{3′}, —O—C₀₋₁₂ alkyl, —SO₂—R³, —SO₂—NR³R^{3′}, —C₀₋₁₂ alkylene-C₂₋₁₂ heteroaryl, and —C₀₋₁₂ alkylene-C₂₋₁₂ heterocyclyl;
R² is chosen from hydrogen, halogen, haloalkyl, and alkyl; and
each of R³ and R^{3′} is independently hydrogen, haloalkyl, or alkyl.

2. The compound of claim 1, being a compound of formula (I-1): 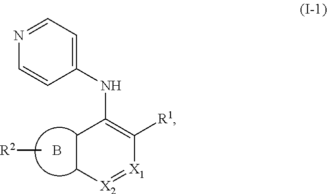
wherein,
X₁ is N or CH;
X₂ is N or CH; and
ring B is aryl or heteroaryl.

3. The compound of claim 2, wherein X₁ and X₂ are CH, or X₁ and X₂ are N.

4. The compound of claim 2, wherein X₁ is CH and X₂ is N, or X₁ is N and X₂ is CH.

5. The compound of claim 2, wherein ring B is an aryl.

6. The compound of claim 5, wherein ring B is phenyl.

7. The compound of claim 2, wherein ring B is a heteroaryl.

8. The compound of claim 7, wherein ring B is a C₂₋₁₂ heteroaryl.

9. The compound of claim 8, wherein ring B is pyridine, furan, or thiophene.

10. The compound of claim 9, wherein ring B is thiophene.

11. The compound of am claim 1, wherein R² is hydrogen.

12. The compound of claim 1, wherein R² is halogen.

13. The compound of claim 12, wherein R² is chloride.

14. The compound of claim 1, wherein the ring A is

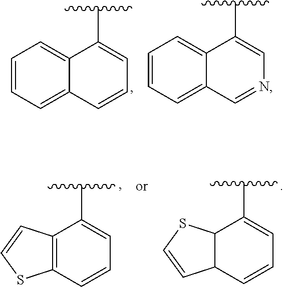

15. The compound of claim 1, wherein R¹ is —C₀₋₆ haloalkyl, —C(O)—C₀₋₆ alkyl, C(O)—C₀₋₆ haloalkyl, —C(O)-aryl, —C(O)-heteroaryl, —C₀₋₆ alkylene-COOH, —CN, —NR³R^{3′}, —N^{3′}—SO₂—R³, —O—C₀₋₆ alkyl, —SO₂—R³, —C₂₋₁₂ heteroaryl, or —C₂₋₁₂ heterocyclyl.

16. The compound of claim 1, wherein R¹ is —CF₂(Me), —C(O)-Me, —COOH, —NH—SO₂-Me, —SO₂-Me, —O-Me, —C(O)—CF₃, —CN, —C(O)-phenyl, —SO₂-Me, —N(CF₃)-Me,

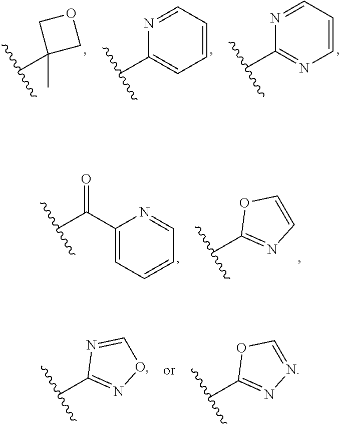

17. A compound selected from the group consisting of: 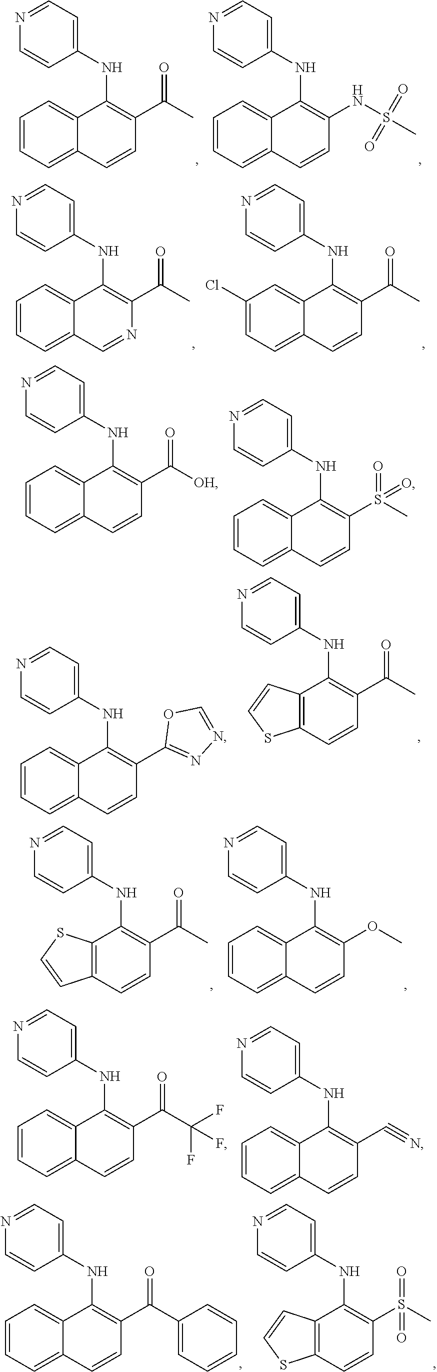 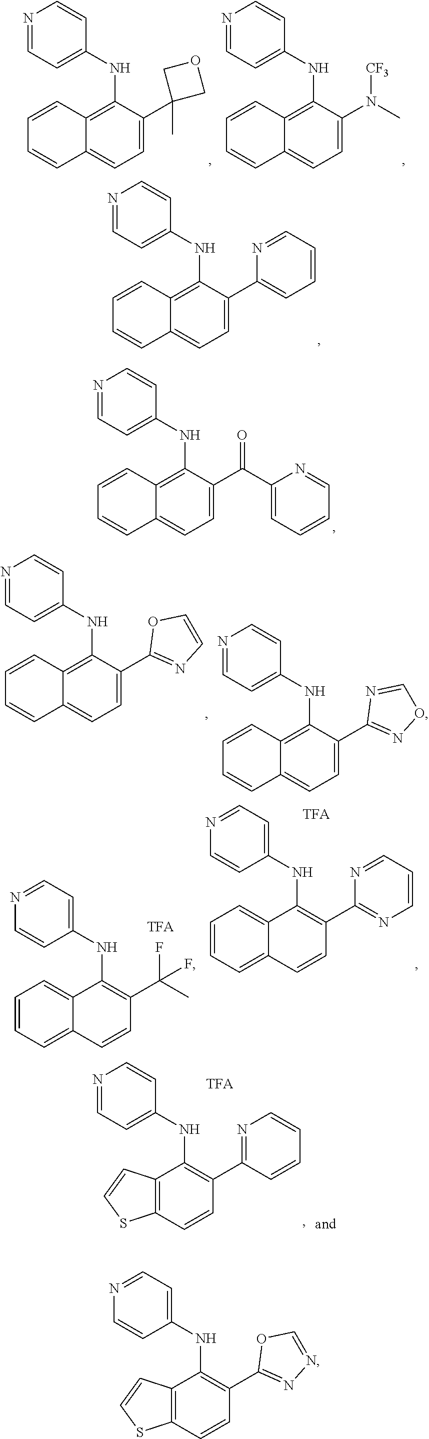
or a stereoisomer, tautomer, pharmaceutically acceptable salt or a solvate thereof.

18. A pharmaceutical composition comprising the compound of claim 1, and a pharmaceutically acceptable carrier.

19. A method of treating or preventing a disease in a subject in need thereof, the method comprising administering to the subject an effective amount of a selective alpha-2C adrenergic receptor (ADRA2C) agonist.

20. The method of claim 19, wherein the disease is nasal congestion, chronic rhinitis, neuropathic pain, inflammatory pain, Raynaud's phenomenon, hypotensive shock, cognitive impairment and neurodegenerative diseases (e.g., Alzheimer's disease, Parkinson's disease, Huntington's disease, amyotrophic lateral sclerosis (ALS), multiple sclerosis (MS), ADHD), schizophrenia, alcohol and drug withdrawal syndrome, rosacea, overactive bladder (OAB), glaucoma, or potential tumor growth modulation.

21. The method of claim 19, wherein the disease is one of TNF-α-mediated inflammatory and autoimmune diseases, such as rheumatoid arthritis, inflammatory bowel disease (IBDS), Crohn's disease, psoriasis, ankylosing spondylitis, sepsis and septic inflammation, acute lung injury (ALI) and acute respiratory distress syndrome (ARDS).

22. The method of claim 19, wherein the selective ADRA2C agonist has an ESR (alpha-2A/alpha-2C) higher than 5, 10, 20, 40, 100, 200, 400, or 800.

23. The method of claim 19, wherein the selective ADRA2C agonist is an alpha-2A adrenergic receptor (ADRA2A) antagonist.

24. The method of claim 19, wherein the selective alpha-2C adrenergic receptor (ADRA2C) agonist is the compound of claim 1.

25. A method of selectively activating alpha-2C adrenergic receptor in a subject in need thereof, the method comprising administering to the subject an effective amount of a selective alpha-2C adrenergic receptor (ADRA2C) agonist.

26. A method of down-regulating TNF-α in a subject in need thereof, the method comprising administering to the subject an effective amount of a selective alpha-2C adrenergic receptor (ADRA2C) agonist.

27. The method of claim 25, wherein the selective alpha-2C adrenergic receptor (ADRA2C) agonist is the compound of claim 1.
